(19) United States
(12) Patent Application Publication
(10) Pub. No.: US 2026/0364344 A1
(43) Pub. Date: Nov. 19, 2026

(54) WEARABLE DEVICES, SYSTEMS, AND METHODS FOR ASSESSING AND/OR MODULATING NEUROLOGICAL CONDITIONS

(71) Applicant: Shuk Ming Maggie POON, Hong Kong (CN)

(72) Inventor: Shuk Ming Maggie POON, Hong Kong (CN)

(21) Appl. No.: 19/647,897

(22) Filed: Apr. 15, 2026

Related U.S. Application Data
(60) Provisional application No. 63/789,435, filed on Apr. 16, 2025.

(51) Int. Cl. A61N 1/20; A61N 1/04

(52) U.S. Cl. CPC A61N 1/20; A61N 1/0472

(57) ABSTRACT

Provided is a wearable device for assessing and/or modulating a neurological condition in a subject, comprising a headset frame, an EEG module and a tDCS module, when the headset frame is worn on the head of the subject, the EEG electrodes are positioned proximate to or in contact with the first region of the subject, and the tDCS electrodes are positioned proximate to or in contact with a second region of the subject. Other example embodiments are described herein. In some embodiments, the wearable devices have a compact, portable and simple design and are sized and shaped for accommodating different sizes and shapes of a subject's head while the EEG electrodes and the tDCS electrodes can accurately and efficiently target the desired target regions of the subject.

### RELATED APPLICATION

This application claims benefit of U.S. Provisional Application having Ser. No. 63/789,435, filed on Apr. 16, 2025, the entire contents of which is/are hereby incorporated by reference herein.

### FIELD OF INVENTION

This invention generally relates to devices, systems and methods for the assessment and modulation of neurological conditions in subjects. More particularly, it concerns devices, systems and methods for AI-based integrated diagnosis and therapeutic intervention of cognitive and neurophysiological conditions using personalized non-invasive brain stimulation techniques (NIBS).

### BACKGROUND OF INVENTION

While mood symptoms in depression are generally well managed, cognitive impairments (COI) remain underdiagnosed and untreated.

Existing assessment tools rely on qualitative measures, lacking diagnostic utility and standardization. Furthermore, there are no clinical guidelines on how to manage cognitive symptoms and patients are often subject to long wait times. As a result, about 45% of cognitive symptoms, potentially more, remain uncontrolled in the existing treatment paradigm. There is a need for devices and systems that can improve the diagnosis and management of cognitive impairments.

### SUMMARY OF INVENTION

In the light of the foregoing background, alternate solutions for devices, systems and methods for assessing and modulating neurological conditions in subjects are provided.

Accordingly an example embodiment of the present disclosure provides a wearable device for assessing and modulating a neurological condition in a subject, including: (a) a headset frame; (b) at least one electroencephalography (EEG) module; (c) at least one transcranial direct current stimulation (tDCS) module; and (d) a wireless communication module for communicating with a remote server, wherein at least one EEG module, the at least one tDCS module, and the wireless communication module are disposed on or housed within the headset frame and are in communication connection with each other, wherein the at least one EEG module is configured to obtain at least one set of EEG data of the subject wearing the wearable device, wherein the wireless communication module is configured to transmit the at least one set of EEG data to the remote server for analysis and receive a tDCS protocol from the remote server, wherein the tDCS protocol is determined based on the analysis of the at least one set of EEG data, and wherein the at least one tDCS module is configured to stimulate at least one target brain region of the subject wearing the wearable device according to the tDCS protocol.

Another example embodiment of the present disclosure provides a wearable device for assessing and modulating a neurological condition in a subject, including: a headset frame; at least one electroencephalography (EEG) modules; at least one transcranial direct current stimulation (tDCS) modules; and a wireless communication module for communicating with a remote server, wherein the at least one EEG module, the at least one tDCS module, and the wireless communication module are disposed on or housed within the headset frame and are in communication connection with each other, wherein the at least one EEG module is configured to obtain at least one set of EEG data of the subject wearing the wearable device, wherein the wireless communication module is configured to transmit the at least one set of EEG data to the remote server for analysis and receive a tDCS protocol from the remote server, wherein the tDCS protocol is determined based on the analysis of the at least one set of EEG data, wherein the at least one tDCS module is configured to stimulate at least one target brain region of the subject wearing the wearable device according to the tDCS protocol, wherein the at least one tDCS module includes a plurality of tDCS electrodes, each configured to stimulate at least a portion of the target brain region of the subject with a current of magnitude around 1.5 mA to around 2 mA, wherein the headset frame is sized and shaped to generally conform to contour of the subject's head portion, wherein the headset frame further includes: a center portion sized and shaped to rest on at least a portion of top of the subject's head, the center portion includes a first side and a second side opposite to the first side; a first arm portion extending from the first side, the first arm portion includes a first arm proximal portion and a first arm bending portion sized and shaped to generally conform to the shape of the subject's right ear; and a second arm portion extending from the second side, the second arm portion includes a second arm proximal portion and a second arm bending portion sized and shaped to generally conform to the shape of the subject's left ear, wherein the at least one tDCS module includes a plurality of tDCS electrodes disposed on interior side of the second arm proximate portion, such that the plurality of tDCS electrodes are proximate to F3 region of the brain of the subject when the wearable device is worn by the subject, wherein the at least one EEG module includes a plurality of EEG electrodes disposed on the interior side of the first arm bending portion such that the plurality of EEG electrodes are proximate to T4 region of the brain of the subject when the wearable device is worn by the subject, wherein the neurological condition includes cognitive impairment (COI), anxiety, depression and/or Attention deficit hyperactivity disorder (ADHD), wherein the wearable device further includes a processing unit configured to analyze the at least one set of EEG data and determine the tDCS protocol, wherein the wearable device further includes a heart rate sensor module configured to obtain at least one set of heart rate data of the subject, wherein the wearable device further includes a rechargeable battery and optionally a battery charging port, wherein the wearable device further includes a wear status module configured to determine a wear status of the wearable device, wherein the wear status includes an on-head status and an off-head status of a subject, and wherein the wear status module includes an infra-red sensor.

Another example embodiment of the present disclosure provides a system for assessing and modulating a neurological condition in a subject, including: (a) at least one wearable device of any one of the preceding claims; (b) a server that is in communication connection with the at least one wearable device via the wireless communication module, including: an artificial intelligence (AI) module configured to: (i) analyze the at least one set of EEG data obtained from the at least one EEG module of each wearable device to generate at least one assessment of the neurological condition of the subject wearing the wearable device; and (ii) generate a tDCS protocol based on the at least one assessment such that the tDCS protocol is customized according to the neurological condition of the subject; (iii) wherein the server is configured to transmit the tDCS protocol to the wearable device, and the at least one tDCS module is configured to simulate the target brain region of the subject according to the tDCS protocol.

Another example embodiment of the present disclosure provides a method for assessing and modulating a neurological condition in a subject using the system of any one of the preceding claims, comprising the steps of: obtaining, by the at least one EEG module, at least one set of EEG data of the subject wearing the wearable device; transmitting, by the wireless communication module of the wearable device, the at least one set of EEG data to the server; analyzing, by the AI module, the at least one set of EEG data to generate at least one assessment of neurological condition of the subject; generating, by the AI module, the tDCS protocol based on the at least one assessment such that the tDCS protocol is customized according to the neurological condition of the subject; transmitting, by the server, the tDCS protocol to the wireless communication module of the wearable device; and executing, by the at least one tDCS module, the tDCS protocol to simulate the target brain region of the subject.

Another example embodiment of the present disclosure provides a wearable device for assessing and/or modulating a neurological condition in a subject, comprising: (a) a headset frame; (b) an electroencephalography (EEG) module, comprising at least one EEG electrodes; and (c) a transcranial direct current stimulation (tDCS) module, comprising at least one tDCS electrodes, wherein the EEG module is configured to obtain at least one set of EEG data of the subject; wherein the tDCS module is configured to stimulate at least one target brain region of the subject; and wherein the headset frame further comprises: (a1) a headband piece, comprising a central portion, a first arm portion and a second arm portion, the headband piece that is sized and shaped to at least span around a first region the subject's head, wherein the central portion is configured to hold the at least one EEG electrodes; (a2) a tDCS electrode holding piece, configured to hold the at least one tDCS electrodes, and wherein, when the headset frame is worn on the head of the subject, the least one EEG electrodes are positioned proximate to or in contact with the first target region, and the at least one tDCS electrodes are positioned proximate to or in contact with a second target region of the subject's head.

Another example embodiment of the present disclosure provides a wearable device for assessing and/or modulating a neurological condition in a subject, comprising: (a) a headset frame; (b) an electroencephalography (EEG) module, comprising two EEG electrodes; and (c) a transcranial direct current stimulation (tDCS) module, comprising three tDCS electrodes; (d) a wireless communication module, for communicating with at least one terminal and optionally at least one remote server; (f) a rechargeable battery and optionally a battery charging port, in electrical connection with EEG module and tDCS module; and (h) a processing unit configured to analyze the at least one set of EEG data and determine a tDCS protocol, wherein the EEG module is configured to obtain at least one set of EEG data of the subject; wherein the tDCS module is configured to stimulate at least one target brain region of the subject; wherein the wireless communication module is configured to transmit the at least one set of EEG data to a terminal or a remote server for analysis and configured to receive a tDCS protocol from the terminal or the remote server; and wherein the headset frame further comprises: (a1) a headband piece, comprising a central portion, a first arm portion, an opposing, second arm portion and a flexible, EEG supporting band extended from or attached to inner surface of the central portion, and the three EEG electrodes are positioned at the EEG supporting band, the headband piece that is sized and shaped to span at least around the T4 region and/or FP1 region the subject's head, wherein the central portion is configured to hold the three EEG electrodes; (a2) a tDCS electrode holding piece, comprising a lateral electrode holding portion and a neck portion, the tDCS electrode holding piece is configured to hold the two tDCS electrodes, wherein the neck portion is integral with or attached to the central portion at the lower side and is integral with or attached to the lateral electrode holding portion at the upper side; wherein the neck portion is configured to be switchable at least between a retracted position and an extended position; wherein, when the headset frame is worn on the head of the subject, the least one EEG electrodes are positioned proximate to or in contact with the FP1 region and/or FP2 region of the subject, and the at least one tDCS electrodes are positioned proximate to or in contact with F3 region and/or F4 region of the subject's head; wherein each of the tDCS electrodes comprises a plurality of conductive silicone electrodes arranged to form a comb-shaped or brush-shaped configuration each of the two tDCS electrodes comprises a plurality of conductive silicone electrodes arranged to form a comb-shaped or brush-shaped configuration, the two TDCS electrodes that are positioned at or proximate to each end of the lateral electrode holding portion and are slidably movable relative to the lateral electrode holding portion; and wherein the tDCS protocol is determined based on the analysis of the at least one set of EEG data, and the at least one tDCS module is configured to stimulate the at least one target brain region of the subject wearing the wearable device according to the tDCS protocol, and each comb-shaped or brush-shaped conductive silicone electrode configured to stimulate at least a portion of the target brain region of the subject with a current of magnitude around 1.5 mA to around 2 mA.

Another example embodiment of the present disclosure provides a system for assessing and/or modulating a neurological condition in a subject, comprising: (a) the at least one wearable device of the present disclosure; (b)at least one terminal; and (c) optionally, at least one remote server, wherein the at least one terminal and optionally the at least one remote server are in communication connection with each of the at least one wearable device, wherein the system is configured to: (i) analyze at least one set of EEG data obtained from the EEG module of the wearable device to generate at least one assessment of the neurological condition of the subject wearing the wearable device; and/or (ii) generate a tDCS protocol based on the at least one assessment, such that the tDCS protocol is customized according to the neurological condition of the subject, wherein the tDCS module is configured to simulate at least one target brain region of the subject according to the tDCS protocol.

Another example embodiment of the present disclosure provides a method for assessing and/or modulating a neurological condition in a subject, comprising the steps of: (a) obtaining, by at least one EEG module, at least one set of EEG data of the subject wearing the wearable device of the present disclosure; (b) optionally, transmitting, by a wireless communication module of the wearable device, the at least one set of EEG data to a terminal or a remote server; (c) analyzing, by the at least one wearable device, the at least one terminal and/or the at least one remote server, the at least one set of EEG data to generate at least one assessment of the neurological condition of the subject; (d) generating, by the at least one wearable device, at least one terminal and/or the at least one remote server, a tDCS protocol based on the at least one assessment such that the tDCS protocol is customized according to the neurological condition of the subject; (e) optionally, transmitting, by the at least one terminal and/or the at least one remote server, the tDCS protocol to the wireless communication module of the wearable device; and (f) executing, by the tDCS module, the tDCS protocol to stimulate at least one target brain region of the subject.

Other example embodiments will be described herein.

There are many advantages of the various embodiments in the present disclosure. In some embodiments, the provided devices, systems, and methods were able to:
(i) quantify cognition using electroencephalogram (EEG);
(ii) identify EEG biomarkers indicative of cognitive impairment (COI); and
(iii) apply a personalized Transcranial Direct Current Stimulation (tDCS) protocol targeting these biomarkers using Artificial Intelligence (AI) technologies.

In some embodiments, provided is a closed-loop system with (i) wireless hybrid EEG-tDCS electrodes for simultaneous EEG recording and tDCS delivery, and (ii) an AI algorithm that detects EEG anomalies and adjusts treatment intensity in real time. Data on biomarkers and treatment will be securely shared with physicians, enabling remote monitoring. In some embodiments, by enhancing cognitive performance in subjects suffering from depression, the provided devices, systems, and methods improve the adherence and efficacy of antidepressants, which are often hindered by poor working memory.

In some embodiments, the provided devices can be used as at-home medical devices to combat the underdiagnosis and undertreatment of cognitive symptoms in subjects suffering from depression.

In some embodiments, the provided devices, systems, and methods can be used by depressive subjects to prevent relapse.

In some embodiments, the provided devices have a compact, portable and simple design and are sized and shaped for accommodating different sizes and shapes of a subject's head while the EEG electrodes and the tDCS electrodes can accurately and efficiently target the desired target regions of the subject.

### BRIEF DESCRIPTION OF FIGURES

FIG. 1A is a top view of a wearable device for assessing and modulating a neurological condition in a subject according to an example embodiment.

FIG. 1B is a perspective view of the wearable device of the same example embodiment as shown in FIG. 1A.

FIG. 1C is a front view of the wearable device of the same example embodiment as shown in FIG. 1A.

FIG. 1D is a right side view of the wearable device of the same example embodiment as shown in FIG. 1A.

FIG. 2A is a front view of an example wearable device worn by a subject.

FIG. 2B is a top view of an example wearable device worn by a subject according to the same example embodiment as shown in FIG. 2A.

FIG. 2C is a perspective view of an example wearable device worn by a subject according to the same example embodiment as shown in FIG. 2A.

FIG. 3 is a schematic diagram showing an example of the corresponding brain positions recorded by the EEG electrodes and stimulated by the tDCS electrodes respectively when the subject is wearing the wearable device.

FIG. 4 is a schematic diagram of an example wearable device.

FIG. 5 is a schematic diagram of an another example wearable device.

FIG. 6 is a circuit diagram of an example wearable device.

FIG. 7 is a circuit diagram of an example wearable device including the tDCS module.

FIG. 8A is a perspective view of another example wearable device for assessing and modulating a neurological condition in a subject according to an example embodiment.

FIG. 8B is a top view of the wearable device of the same example embodiment as shown in FIG. 8A.

FIG. 8C is a bottom view of the wearable device of the same example embodiment as shown in FIG. 8A.

FIG. 8D is a side view of the wearable device of the same example embodiment as shown in FIG. 8A.

FIG. 8E is a side view of the example wearable device of FIG. 8A worn by a subject.

FIG. 8F is a perspective view of an example wearable device worn by a subject according to the same example embodiment as shown in FIG. 8A.

FIG. 8G is a front view of an example wearable device worn by a subject according to the same example embodiment as shown in FIG. 8A.

FIG. 9 is a schematic diagram of an example system for assessing and/or modulating a neurological condition in a subject.

FIG. 10 is a schematic diagram of an example method for assessing and/or modulating a neurological condition in a subject using an example system

### DETAILED DESCRIPTION

In some embodiments, provided is a wearable device for assessing and modulating a neurological condition in a subject, including: (a) a headset frame; (b) at least one electroencephalography (EEG) module; (c) at least one transcranial direct current stimulation (tDCS) module; and (d) a wireless communication module for communicating with a remote server, wherein at least one EEG module, the at least one tDCS module, and the wireless communication module are disposed on or housed within the headset frame and are in communication connection with each other, wherein the at least one EEG module is configured to obtain at least one set of EEG data of the subject wearing the wearable device, wherein the wireless communication module is configured to transmit the at least one set of EEG data to the remote server for analysis and receive a tDCS protocol from the remote server, wherein the tDCS protocol is determined based on the analysis of the at least one set of EEG data, and wherein the at least one tDCS module is configured to stimulate at least one target brain region of the subject wearing the wearable device according to the tDCS protocol.

In some embodiments, the at least one tDCS module includes a plurality of tDCS electrodes, each configured to stimulate at least a portion of the target brain region of the subject with a current of magnitude around 1.5 mA to around 2 mA.

In some embodiments, the headset frame is sized and shaped to generally conform to contour of the subject's head portion, and wherein the headset frame further includes: a center portion sized and shaped to rest on at least a portion of top of the subject's head, the center portion includes a first side and a second side opposite to the first side; a first arm portion extending from the first side, the first arm portion includes a first arm proximal portion and a first arm bending portion sized and shaped to generally conform to the shape of the subject's right ear; and a second arm portion extending from the second side, the second arm portion includes a second arm proximal portion and a second arm bending portion sized and shaped to generally conform to the shape of the subject's left ear.

In some embodiments, the at least one tDCS module includes a plurality of tDCS electrodes disposed on interior side of the second arm proximate portion, such that the plurality of tDCS electrodes are proximate to F3 region of the brain of the subject when the wearable device is worn by the subject.

In some embodiments, the at least one EEG module includes a plurality of EEG electrodes disposed on the interior side of the first arm bending portion such that the plurality of EEG electrodes are proximate to T4 region of the brain of the subject when the wearable device is worn by the subject.

In some embodiments, the neurological condition includes cognitive impairment (COI), anxiety, depression and/or Attention deficit hyperactivity disorder (ADHD).

In some embodiments, the wearable device further includes a processing unit configured to analyze the at least one set of EEG data and determine the tDCS protocol.

In some embodiments, the wearable device further includes a heart rate sensor module configured to obtain at least one set of heart rate data of the subject.

In some embodiments, the wearable device further includes a rechargeable battery and optionally a battery charging port.

In some embodiments, the wearable device further includes a wear status module configured to determine a wear status of the wearable device, wherein the wear status includes an on-head status and an off-head status of a subject.

In some embodiments, the wear status module includes an infra-red sensor.

In some embodiments, provided is a wearable device for assessing and modulating a neurological condition in a subject, including: a headset frame; at least one electroencephalography (EEG) modules; at least one transcranial direct current stimulation (tDCS) modules; and a wireless communication module for communicating with a remote server, wherein the at least one EEG module, the at least one tDCS module, and the wireless communication module are disposed on or housed within the headset frame and are in communication connection with each other, wherein the at least one EEG module is configured to obtain at least one set of EEG data of the subject wearing the wearable device, wherein the wireless communication module is configured to transmit the at least one set of EEG data to the remote server for analysis and receive a tDCS protocol from the remote server, wherein the tDCS protocol is determined based on the analysis of the at least one set of EEG data, wherein the at least one tDCS module is configured to stimulate at least one target brain region of the subject wearing the wearable device according to the tDCS protocol, wherein the at least one tDCS module includes a plurality of tDCS electrodes, each configured to stimulate at least a portion of the target brain region of the subject with a current of magnitude around 1.5 mA to around 2 mA, wherein the headset frame is sized and shaped to generally conform to contour of the subject's head portion, wherein the headset frame further includes: a center portion sized and shaped to rest on at least a portion of top of the subject's head, the center portion includes a first side and a second side opposite to the first side; a first arm portion extending from the first side, the first arm portion includes a first arm proximal portion and a first arm bending portion sized and shaped to generally conform to the shape of the subject's right ear; and a second arm portion extending from the second side, the second arm portion includes a second arm proximal portion and a second arm bending portion sized and shaped to generally conform to the shape of the subject's left ear, wherein the at least one tDCS module includes a plurality of tDCS electrodes disposed on interior side of the second arm proximate portion, such that the plurality of tDCS electrodes are proximate to F3 region of the brain of the subject when the wearable device is worn by the subject, wherein the at least one EEG module includes a plurality of EEG electrodes disposed on the interior side of the first arm bending portion such that the plurality of EEG electrodes are proximate to T4 region of the brain of the subject when the wearable device is worn by the subject, wherein the neurological condition includes cognitive impairment (COI), anxiety, depression and/or Attention deficit hyperactivity disorder (ADHD), wherein the wearable device further includes a processing unit configured to analyze the at least one set of EEG data and determine the tDCS protocol, wherein the wearable device further includes a heart rate sensor module configured to obtain at least one set of heart rate data of the subject, wherein the wearable device further includes a rechargeable battery and optionally a battery charging port, wherein the wearable device further includes a wear status module configured to determine a wear status of the wearable device, wherein the wear status includes an on-head status and an off-head status of a subject, and wherein the wear status module includes an infra-red sensor.

In some embodiments, provided is a system for assessing and modulating a neurological condition in a subject, including: (a) at least one wearable device of any one of the preceding claims; (b) a server that is in communication connection with the at least one wearable device via the wireless communication module, including: an artificial intelligence (AI) module configured to: (i) analyze the at least one set of EEG data obtained from the at least one EEG module of each wearable device to generate at least one assessment of the neurological condition of the subject wearing the wearable device; and (ii) generate a tDCS protocol based on the at least one assessment such that the tDCS protocol is customized according to the neurological condition of the subject; (iii) wherein the server is configured to transmit the tDCS protocol to the wearable device, and the at least one tDCS module is configured to simulate the target brain region of the subject according to the tDCS protocol.

In some embodiments, the server is a cloud-based platform connected wirelessly to the at least one wearable device via the wireless communication module.

In some embodiments, the neurological condition includes cognitive impairment (COI), anxiety, depression and/or Attention deficit hyperactivity disorder (ADHD).

In some embodiments, provided is a method for assessing and modulating a neurological condition in a subject using the system, including the steps of: (a) obtaining, by the at least one EEG module, at least one set of EEG data of the subject wearing the wearable device; (b) transmitting, by the wireless communication module of the wearable device, the at least one set of EEG data to the server; (c) analyzing, by the AI module, the at least one set of EEG data to generate at least one assessment of neurological condition of the subject; (d) generating, by the AI module, the tDCS protocol based on the at least one assessment such that the tDCS protocol is customized according to the neurological condition of the subject; (e) transmitting, by the server, the tDCS protocol to the wireless communication module of the wearable device; and (f) executing, by the at least one tDCS module, the tDCS protocol to simulate the target brain region of the subject.

In some embodiments, the method further includes the following steps prior to step (a): (i) obtaining at least one set of training EEG data from a reference data source labeled by a set of neurological conditions and/or a set of tDCS protocols; (ii) training the AI module with the at least one set of training EEG data until a performance of the AI module meets a predetermined performance condition; and (iii) (iv) deploying the AI module for performing steps (c) and/or (d).

In some embodiments, the method further includes the step of: (g) obtaining, by a heart rate sensor module, at least one heart rate data of the subject.

In some embodiments, the step (f) further includes the step of: (f1) stimulating, by the at least one tDCS module, the target brain region of the subject with a current of magnitude around 1.5 mA to around 2 mA.

### Definitions

As used herein and in the claims, the terms “comprising” (or any related form such as “comprise” and “comprises”), “including” (or any related forms such as “include” or “includes”), “containing” (or any related forms such as “contain” or “contains”), means including the following elements but not excluding others. It shall be understood that for every embodiment in which the term “comprising” (or any related form such as “comprise” and “comprises”), “including” (or any related forms such as “include” or “includes”), or “containing” (or any related forms such as “contain” or “contains”) is used, this disclosure/application also includes alternate embodiments where the term “comprising”, “including,” or “containing,” is replaced with “consisting essentially of” or “consisting of”. These alternate embodiments that use “consisting of” or “consisting essentially of” are understood to be narrower embodiments of the “comprising”, “including,” or “containing,” embodiments.

For the sake of clarity, “comprising”, “including”, “containing” and “having”, and any related forms are open-ended terms which allows for additional elements or features beyond the named essential elements, whereas “consisting of” is a closed end term that is limited to the elements recited in the claim and excludes any element, step, or ingredient not specified in the claim.

As used herein, the singular forms “a”, “an” and “the” are intended to include the plural forms as well, unless the context clearly indicates otherwise. Where a range is referred in the specification, the range is understood to include each discrete point within the range. For example, 1-7 means 1, 2, 3, 4, 5, 6, and 7.

As used herein and in the claims, the term “about” is understood as within a range of normal tolerance in the art and not more than ±10% of a stated value. By way of example only, about 50 means from 45 to 55, including all values in between. As used herein, the phrase “about” a specific value also includes the specific value, for example, “about 50” includes 50.

As used herein and in the claims, the terms “general” or “generally”, or “substantial” or “substantially” mean that the recited characteristic, angle, shape, state, structure, or value need not be achieved exactly, but that deviations or variations, including for example, tolerances, measurement error, measurement accuracy limitations and other factors known to those of skill in the art, may occur in amounts that do not preclude the effect the characteristic was intended to provide. For example, an object that has a “generally” cylindrical shape would mean that the object has either an exact cylindrical shape or a nearly exact cylindrical shape. In another example, an object that is “substantially” perpendicular to a surface would mean that the object is either exactly perpendicular to the surface or nearly exactly perpendicular to the surface, e.g., has a 5% deviation.

It is to be understood that terms such as “top”, “bottom”, “middle”, “side”, “length”, “inner”, “outer”, “interior”, “exterior”, “outside”, “vertical”, “horizontal” and the like as may be used herein, merely describe points of reference and do not limit the present invention to any particular orientation or configuration. Further, terms such as “first,” “second,” “third,” etc., merely identify one of a number of portions, components and/or points of reference as disclosed herein, and likewise do not limit the present invention to any particular configuration or orientation.

As used herein and in the claims, “couple” or “connect” refers to electrical coupling or connection either directly or indirectly via one or more electrical means unless otherwise stated.

As used herein and in the claims, “electroencephalogram” or “EEG” refers to a non-invasive neurophysiological monitoring technique that records the electrical activity of the brain through electrodes placed on the scalp of a subject.

As used herein and in the claims, “Transcranial Direct Current Stimulation” or “tDCS” refers to non-invasive neuromodulation technique that delivers a low-intensity, constant electrical current through electrodes placed on the scalp of a subject to modulate neuronal excitability in targeted brain regions.

As used herein and in the claims, “cognitive impairment” or “COI” refers to a decline or dysfunction in one or more cognitive domains, including memory, attention, executive function, language, visuospatial skills, and processing speed, which may interfere with an individual's ability to perform everyday activities.

As used herein and in the claims, “operatively connect” or “operatively connected” refers to a connection between two or more components that enables them to function together in an intended manner, including but not limited to the transmission of data, signals, or power between the components. In some examples, an operative connection is achieved through direct physical contact, indirect connection via one or more intermediate components, or through wired or wireless communication pathways.

As used herein and in the claims, “in electrical communication” refers to a connection between two or more components through which electrical signals, electrical power, or both may be transmitted. In some examples, components in electrical communication are directly connected or indirectly connected via one or more intermediate electrical conductors, circuits, or components.

As used herein and in the claims, “in wireless communication” refers to a connection between two or more components or devices that enables the transmission of data or signals without the use of physical wired connections. In some examples, wireless communication is achieved through radio frequency technologies such as Bluetooth, Wi-Fi, cellular networks, or other electromagnetic transmission methods.

As used herein and in the claims, a “subject” refers to animals such as mammals, including, but not limited to, primates (e.g., humans), cows, sheep, goats, horses, dogs, cats, rabbits, rats, mice and the like. In some examples, the subject refers to human.

As used herein and in the claims, “wearable device” refers to an apparatus configured to be worn on a subject. In this example, the wearable device is worn on a subject's head for assessing and/or modulating a neurological condition, comprising at least a headset frame, an EEG module, and a tDCS module.

As used herein and in the claims, “system” refers to an integrated arrangement of components comprising at least one wearable device, at least one terminal, and optionally at least one remote server, configured to work together for assessing and/or modulating a neurological condition in a subject.

As used herein and in the claims, “method” refers to a series of steps or procedures performed for assessing and/or modulating a neurological condition in a subject using a wearable device or system as described herein.

As used herein and in the claims, “neurological condition” refers to a disorder or dysfunction affecting the brain or nervous system, including but not limited to cognitive impairment, anxiety, depression, and attention deficit hyperactivity disorder.

As used herein and in the claims, “anxiety” refers to a neurological or psychological condition characterized by excessive worry, nervousness, or unease, which may be assessed and/or modulated using the devices, systems, and methods described herein.

As used herein and in the claims, “depression” refers to a mood disorder characterized by persistent feelings of sadness, loss of interest, or other emotional and cognitive symptoms, which may be assessed and/or modulated using the devices, systems, and methods described herein.

As used herein and in the claims, “attention deficit hyperactivity disorder” or “ADHD” refers to a neurodevelopmental disorder characterized by symptoms of inattention, hyperactivity, and impulsivity, which may be assessed and/or modulated using the devices, systems, and methods described herein.

As used herein and in the claims, “headset frame” refers to a structural housing or arcuate frame that is sized and shaped to conform to the contour of a subject's head and configured to support and hold the EEG module, tDCS module, and other components of the wearable device.

As used herein and in the claims, “EEG module” refers to a component of the wearable device comprising at least one EEG electrode and configured to obtain at least one set of EEG data from the subject, and may further include signal amplifiers, digital converters, and microcontrollers for data processing.

As used herein and in the claims, “EEG electrode” or “EEG electrodes” refers to one or more sensors configured to be placed on or proximate to the scalp of a subject to detect and record electrical activity of the brain.

As used herein and in the claims, “EEG data” refers to electrical signals or information recorded from the brain of a subject through EEG electrodes, which may include measurements of absolute spectral power at certain frequency bands and may be used to assess neurological conditions.

As used herein and in the claims, “tDCS module” refers to a component of the wearable device comprising at least one tDCS electrode and configured to deliver transcranial direct current stimulation to at least one target brain region of the subject, and may further include control and safety circuits.

As used herein and in the claims, “tDCS electrode” or “tDCS electrodes” refers to one or more electrodes configured to deliver low-intensity electrical current to the scalp of a subject for stimulating target brain regions, and may include at least one anode and one cathode.

As used herein and in the claims, “target brain region” refers to a specific area of the brain intended to be monitored by EEG electrodes and/or stimulated by tDCS electrodes, such as the Dorsolateral Prefrontal Cortex or other regions identified according to the International 10-20 system.

As used herein and in the claims, “first region” refers to a region of the subject's head where the EEG electrodes are positioned when the wearable device is worn, which may include the T4 region, FP1 region, and/or FP2 region.

As used herein and in the claims, “first target region” refers to the area of the subject's head where the EEG electrodes are positioned proximate to or in contact with when the headset frame is worn, for obtaining EEG data.

As used herein and in the claims, “second target region” refers to the area of the subject's head where the tDCS electrodes are positioned proximate to or in contact with when the headset frame is worn, for delivering transcranial direct current stimulation.

As used herein and in the claims, “T4 region” refers to a location on the scalp corresponding to the right temporal area of the brain according to the International 10-20 electrode placement system.

As used herein and in the claims, “FP1 region” refers to a location on the scalp corresponding to the left frontal pole area of the brain according to the International 10-20 electrode placement system.

As used herein and in the claims, “FP2 region” refers to a location on the scalp corresponding to the right frontal pole area of the brain according to the International 10-20 electrode placement system.

As used herein and in the claims, “F3 region” refers to a location on the scalp corresponding to the left frontal area of the brain according to the International 10-20 electrode placement system.

As used herein and in the claims, “F4 region” refers to a location on the scalp corresponding to the right frontal area of the brain according to the International 10-20 electrode placement system.

As used herein and in the claims, “headband piece” refers to a component of the headset frame comprising a central portion, a first arm portion, and a second arm portion, sized and shaped to span around at least a portion of the subject's head.

As used herein and in the claims, “central portion” refers to a part of the headband piece configured to rest on or proximate to the forehead or top of the subject's head and configured to hold the EEG electrodes.

As used herein and in the claims, “first arm portion” refers to a part of the headband piece that extends from a first side of the central portion and is configured to extend posteriorly along one side of the subject's head.

As used herein and in the claims, “second arm portion” refers to a part of the headband piece that extends from a second side of the central portion, opposite to the first side, and is configured to extend posteriorly along the opposite side of the subject's head.

As used herein and in the claims, “tDCS electrode holding piece” refers to a component of the headset frame configured to hold and support the tDCS electrodes, and may include a lateral electrode holding portion and a neck portion.

As used herein and in the claims, “lateral electrode holding portion” refers to a part of the tDCS electrode holding piece that extends laterally and is configured to hold the tDCS electrodes at or proximate to each of its ends.

As used herein and in the claims, “neck portion” refers to a part of the tDCS electrode holding piece that connects the central portion to the lateral electrode holding portion and is configured to be switchable between at least a retracted position and an extended position.

As used herein and in the claims, “retracted position” refers to a configuration of the neck portion in which it is shortened or contracted, allowing the tDCS electrodes to be positioned closer to the central portion of the headset frame.

As used herein and in the claims, “extended position” refers to a configuration of the neck portion in which it is lengthened or expanded, allowing the tDCS electrodes to be positioned further from the central portion of the headset frame to accommodate different head sizes and shapes.

As used herein and in the claims, “flexible EEG supporting band” refers to a component made of flexible materials, such as silicone, that is extended from or attached to the inner surface of the central portion and configured to hold the EEG electrodes while conforming to the contour of the subject's head.

As used herein and in the claims, “wireless communication module” refers to a component of the wearable device configured to wirelessly transmit and receive data, including EEG data and tDCS protocols, to and from at least one terminal and/or at least one remote server, and may include Wi-Fi and/or Bluetooth capabilities.

As used herein and in the claims, “terminal” refers to a device in communication connection with the wearable device, configured to serve as a user interface for controlling the wearable device and for receiving, transmitting, and/or displaying data, and may include a smartphone, tablet, personal computer, and/or dedicated remote control.

As used herein and in the claims, “remote server” refers to a computing device or platform located remotely from the wearable device and configured to receive EEG data, perform analysis, generate assessments and/or tDCS protocols, and transmit data to the wearable device and/or terminal.

As used herein and in the claims, “cloud-based platform” refers to a remote server that provides computing services over a network, configured to be in wireless communication with the wearable device and/or terminal for data storage, processing, and transmission.

As used herein and in the claims, “processing unit” refers to a component configured to analyze EEG data, determine tDCS protocols, and control the operation of the wearable device, and may include a microprocessor or microcontroller unit.

As used herein and in the claims, “communication connection” refers to a link between two or more components or devices that enables the transmission of data, signals, or control commands, which may be achieved through wired or wireless means.

As used herein and in the claims, “assessment” refers to an evaluation or determination of the neurological condition of a subject based on analysis of EEG data and/or other data obtained from the subject.

As used herein and in the claims, “tDCS protocol” refers to a set of parameters or instructions for delivering transcranial direct current stimulation to a subject, which may be customized based on the assessment of the subject's neurological condition and may include parameters such as current intensity, duration, and target brain regions.

As used herein and in the claims, “heart rate sensor module” refers to a component of the wearable device configured to measure and obtain at least one set of heart rate data from the subject.

As used herein and in the claims, “heart rate data” refers to information relating to the heart rate of the subject obtained by the heart rate sensor module.

As used herein and in the claims, “rechargeable battery” refers to a power source configured to store electrical energy and provide power to the components of the wearable device, and capable of being recharged through a charging interface.

As used herein and in the claims, “battery charging port” refers to an interface on the wearable device configured to receive external power for charging the rechargeable battery, and may include a Type-C port or magnetic charging port.

As used herein and in the claims, “wear status module” refers to a component of the wearable device configured to determine whether the wearable device is being worn by a subject, including detecting an on-head status or an off-head status.

As used herein and in the claims, “wear status” refers to the state of the wearable device with respect to whether it is being worn by a subject, including an on-head status and an off-head status.

As used herein and in the claims, “on-head status” refers to a wear status indicating that the wearable device is positioned on and being worn by the subject's head.

As used herein and in the claims, “off-head status” refers to a wear status indicating that the wearable device is not positioned on or being worn by the subject's head.

As used herein and in the claims, “infra-red sensor” refers to a sensor configured to detect infrared radiation, which may be used in the wear status module to determine whether the wearable device is being worn by a subject.

As used herein and in the claims, “conductive silicone electrode” or “conductive silicone electrodes” refers to one or more electrodes made of electrically conductive silicone material, configured to deliver electrical current to the scalp of a subject for tDCS stimulation.

As used herein and in the claims, “comb-shaped configuration” refers to an arrangement of a plurality of conductive silicone electrodes in which the electrodes are arranged in a pattern resembling the teeth of a comb, configured to facilitate contact with the scalp through hair.

As used herein and in the claims, “brush-shaped configuration” refers to an arrangement of a plurality of conductive silicone electrodes in which the electrodes are arranged in a pattern resembling the bristles of a brush, configured to facilitate contact with the scalp through hair.

As used herein and in the claims, “slidably movable” refers to a configuration in which a component, such as a tDCS electrode or tDCS electrode socket, is capable of being moved along a sliding rail or track relative to another component, such as the lateral electrode holding portion, to allow adjustment of position.

### NUMBERED EMBODIMENTS

### Set 1

Embodiment 1. A wearable device for assessing and modulating a neurological condition in a subject, comprising: (a) a headset frame; (b) at least one electroencephalography (EEG) module; (c) at least one transcranial direct current stimulation (tDCS) module; and (d) a wireless communication module for communicating with a remote server, wherein at least one EEG module, the at least one tDCS module, and the wireless communication module are disposed on or housed within the headset frame and are in communication connection with each other, wherein the at least one EEG module is configured to obtain at least one set of EEG data of the subject wearing the wearable device, wherein the wireless communication module is configured to transmit the at least one set of EEG data to the remote server for analysis and receive a tDCS protocol from the remote server, wherein the tDCS protocol is determined based on the analysis of the at least one set of EEG data, and wherein the at least one tDCS module is configured to stimulate at least one target brain region of the subject wearing the wearable device according to the tDCS protocol.

Embodiment 2. The wearable device of embodiment 1, wherein the at least one tDCS module comprises a plurality of tDCS electrodes, each configured to stimulate at least a portion of the target brain region of the subject with a current of magnitude around 1.5 mA to around 2 mA.

Embodiment 3. The wearable device of any one of the preceding embodiments, wherein the headset frame is sized and shaped to generally conform to contour of the subject's head portion, and wherein the headset frame further comprises: a center portion sized and shaped to rest on at least a portion of top of the subject's head, the center portion comprises a first side and a second side opposite to the first side; a first arm portion extending from the first side, the first arm portion comprises a first arm proximal portion and a first arm bending portion sized and shaped to generally conform to the shape of the subject's right ear; and a second arm portion extending from the second side, the second arm portion comprises a second arm proximal portion and a second arm bending portion sized and shaped to generally conform to the shape of the subject's left ear.

Embodiment 4. The wearable device of embodiment 3, wherein the at least one tDCS module comprises a plurality of tDCS electrodes disposed on interior side of the second arm proximate portion, such that the plurality of tDCS electrodes are proximate to F3 region of the brain of the subject when the wearable device is worn by the subject.

Embodiment 5. The wearable device of embodiment 3, wherein the at least one EEG module comprises a plurality of EEG electrodes disposed on the interior side of the first arm bending portion such that the plurality of EEG electrodes are proximate to T4 region of the brain of the subject when the wearable device is worn by the subject.

Embodiment 6. The wearable device of any one of the preceding embodiments, wherein the neurological condition comprises cognitive impairment (COI), anxiety, depression and/or Attention deficit hyperactivity disorder (ADHD).

Embodiment 7. The wearable device of any one of the preceding embodiments, further comprising a processing unit configured to analyze the at least one set of EEG data and determine the tDCS protocol.

Embodiment 8. The wearable device of any one of the preceding embodiments, further comprising a heart rate sensor module configured to obtain at least one set of heart rate data of the subject.

Embodiment 9. The wearable device of any one of the preceding embodiments, further comprising a rechargeable battery and optionally a battery charging port.

Embodiment 10. The wearable device of any one of the preceding embodiments, further comprising a wear status module configured to determine a wear status of the wearable device, wherein the wear status comprises an on-head status and an off-head status of a subject.

Embodiment 11. The wearable device of embodiment 10, wherein the wear status module comprises an infra-red sensor.

Embodiment 12. A wearable device for assessing and modulating a neurological condition in a subject, comprising: a headset frame; at least one electroencephalography (EEG) modules; at least one transcranial direct current stimulation (tDCS) modules; and a wireless communication module for communicating with a remote server, wherein the at least one EEG module, the at least one tDCS module, and the wireless communication module are disposed on or housed within the headset frame and are in communication connection with each other, wherein the at least one EEG module is configured to obtain at least one set of EEG data of the subject wearing the wearable device, wherein the wireless communication module is configured to transmit the at least one set of EEG data to the remote server for analysis and receive a tDCS protocol from the remote server, wherein the tDCS protocol is determined based on the analysis of the at least one set of EEG data, wherein the at least one tDCS module is configured to stimulate at least one target brain region of the subject wearing the wearable device according to the tDCS protocol, wherein the at least one tDCS module comprises a plurality of tDCS electrodes, each configured to stimulate at least a portion of the target brain region of the subject with a current of magnitude around 1.5 mA to around 2 mA, wherein the headset frame is sized and shaped to generally conform to contour of the subject's head portion, wherein the headset frame further comprises: a center portion sized and shaped to rest on at least a portion of top of the subject's head, the center portion comprises a first side and a second side opposite to the first side; a first arm portion extending from the first side, the first arm portion comprises a first arm proximal portion and a first arm bending portion sized and shaped to generally conform to the shape of the subject's right ear; and a second arm portion extending from the second side, the second arm portion comprises a second arm proximal portion and a second arm bending portion sized and shaped to generally conform to the shape of the subject's left ear, wherein the at least one tDCS module comprises a plurality of tDCS electrodes disposed on interior side of the second arm proximate portion, such that the plurality of tDCS electrodes are proximate to F3 region of the brain of the subject when the wearable device is worn by the subject, wherein the at least one EEG module comprises a plurality of EEG electrodes disposed on the interior side of the first arm bending portion such that the plurality of EEG electrodes are proximate to T4 region of the brain of the subject when the wearable device is worn by the subject, wherein the neurological condition comprises cognitive impairment (COI), anxiety, depression and/or Attention deficit hyperactivity disorder (ADHD), wherein the wearable device further comprises a processing unit configured to analyze the at least one set of EEG data and determine the tDCS protocol, wherein the wearable device further comprises a heart rate sensor module configured to obtain at least one set of heart rate data of the subject, wherein the wearable device further comprises a rechargeable battery and optionally a battery charging port, wherein the wearable device further comprises a wear status module configured to determine a wear status of the wearable device, wherein the wear status comprises an on-head status and an off-head status of a subject, and wherein the wear status module comprises an infra-red sensor.

Embodiment 13. A system for assessing and modulating a neurological condition in a subject, comprising: (a) at least one wearable device of any one of the preceding embodiments; (b) a server that is in communication connection with the at least one wearable device via the wireless communication module, comprising: an artificial intelligence (AI) module configured to: (i) analyze the at least one set of EEG data obtained from the at least one EEG module of each wearable device to generate at least one assessment of the neurological condition of the subject wearing the wearable device; and (ii) generate a tDCS protocol based on the at least one assessment such that the tDCS protocol is customized according to the neurological condition of the subject; wherein the server is configured to transmit the tDCS protocol to the wearable device, and the at least one tDCS module is configured to simulate the target brain region of the subject according to the tDCS protocol.

Embodiment 14. The system of embodiment 13, wherein the server is a cloud-based platform connected wirelessly to the at least one wearable device via the wireless communication module.

Embodiment 15. The system of any one of the preceding embodiments, wherein the neurological condition comprises cognitive impairment (COI), anxiety, depression and/or Attention deficit hyperactivity disorder (ADHD).

Embodiment 16. A method for assessing and modulating a neurological condition in a subject using the system of any one of the preceding embodiments, comprising the steps of: (a) obtaining, by the at least one EEG module, at least one set of EEG data of the subject wearing the wearable device; (b) transmitting, by the wireless communication module of the wearable device, the at least one set of EEG data to the server; (c) analyzing, by the AI module, the at least one set of EEG data to generate at least one assessment of neurological condition of the subject; (d) generating, by the AI module, the tDCS protocol based on the at least one assessment such that the tDCS protocol is customized according to the neurological condition of the subject; (e) transmitting, by the server, the tDCS protocol to the wireless communication module of the wearable device; and (f) executing, by the at least one tDCS module, the tDCS protocol to simulate the target brain region of the subject.

Embodiment 17. The method of embodiment 16, further comprising the following steps prior to step (a): (i) obtaining at least one set of training EEG data from a reference data source labeled by a set of neurological conditions and/or a set of tDCS protocols; (ii) training the AI module with the at least one set of training EEG data until a performance of the AI module meets a predetermined performance condition; and (iii) deploying the AI module for performing steps (c) and/or (d).

Embodiment 18. The method of any one of the preceding embodiments, further comprises the step of: (g) obtaining, by a heart rate sensor module, at least one heart rate data of the subject.

Embodiment 19. The method of any one of the preceding embodiments, wherein step (f) further comprises the step of: (f1) stimulating, by the at least one tDCS module, the target brain region of the subject with a current of magnitude around 1.5 mA to around 2 mA.

### Set 2

Embodiment 1. A wearable device for assessing and/or modulating a neurological condition in a subject, comprising: (a) a headset frame; (b) an electroencephalography (EEG) module, comprising at least one EEG electrodes; and (c) a transcranial direct current stimulation (tDCS) module, comprising at least one tDCS electrodes, wherein the EEG module is configured to obtain at least one set of EEG data of the subject; wherein the tDCS module is configured to stimulate at least one target brain region of the subject; and wherein the headset frame further comprises: (a1) a headband piece, comprising a central portion, a first arm portion and a second arm portion, the headband piece that is sized and shaped to at least span around a first region the subject's head, wherein the central portion is configured to hold the at least one EEG electrodes; (a2) a tDCS electrode holding piece, configured to hold the at least one tDCS electrodes, and wherein, when the headset frame is worn on the head of the subject, the least one EEG electrodes are positioned proximate to or in contact with the first target region, and the at least one tDCS electrodes are positioned proximate to or in contact with a second target region of the subject's head.

Embodiment 2. The wearable device of embodiment 1, wherein the first region is or comprises T4 region, FP1 region and/or FP2 region; and/or the second region is or comprises F3 region and/or F4 region.

Embodiment 3. The wearable device of embodiment 1, wherein the tDCS electrode holding piece comprises a lateral electrode holding portion and a neck portion; wherein the neck portion is integral with or attached to the central portion at the lower side and is integral with or attached to the lateral electrode holding portion at the upper side; and wherein the neck portion is configured to be switchable at least between a retracted position and an extended position.

Embodiment 4. The wearable device of embodiment 1, further comprising a wireless communication module for communicating with at least one terminal and/or at least one remote server.

Embodiment 5. The wearable device of embodiment 4, wherein the wireless communication module is configured to transmit the at least one set of EEG data to the at least one terminal and/or the at least one remote server for analysis and is configured to receive a tDCS protocol from the at least one terminal and/or the at least one remote server; and wherein the tDCS protocol is determined based on the analysis of the at least one set of EEG data.

Embodiment 6. The wearable device of embodiment 5, wherein the at least one tDCS module is configured to stimulate the at least one target brain region of the subject wearing the wearable device according to the tDCS protocol.

Embodiment 7. The wearable device of embodiment 1, further comprising a flexible, EEG supporting band extended from or attached to inner surface of the central portion, and wherein the at least one EEG electrodes comprise three EEG electrodes that are positioned at the EEG supporting band.

Embodiment 8. The wearable device of embodiment 3, wherein the at least one tDCS electrodes comprise two TDCS electrodes that are positioned at or proximate to each end of the lateral electrode holding portion.

Embodiment 9. The wearable device of embodiment 1, wherein each of the tDCS electrodes comprises a plurality of conductive silicone electrodes arranged to form a comb-shaped or brush-shaped configuration.

Embodiment 10. The wearable device of embodiment 3, wherein the at least one tDCS electrodes are configured to be slidably movable relative to the lateral electrode holding portion.

Embodiment 11. The wearable device of embodiment 1, wherein the at least one tDCS module comprises a plurality of tDCS electrodes, each configured to stimulate at least a portion of the target brain region of the subject with a current of magnitude around 1.5 mA to around 2 mA.

Embodiment 12. The wearable device of embodiment 1, wherein the neurological condition comprises cognitive impairment (COI), anxiety, depression and/or attention deficit hyperactivity disorder (ADHD).

Embodiment 13. The wearable device of embodiment 5, further comprising a processing unit configured to analyze the at least one set of EEG data and determine the tDCS protocol.

Embodiment 14. The wearable device of embodiment 1, further comprising a heart rate sensor module configured to obtain at least one set of heart rate data of the subject.

Embodiment 15. The wearable device of embodiment 1, further comprising a rechargeable battery and optionally a battery charging port in electrical connection with the EEG module and the tDCS module.

Embodiment 16. The wearable device of embodiment 1, further comprising a wear status module configured to determine a wear status of the wearable device, wherein the wear status comprising an on-head status and an off-head status of the subject.

Embodiment 17. The wearable device of embodiment 16, wherein the wear status module comprising an infra-red sensor.

Embodiment 18. A wearable device for assessing and/or modulating a neurological condition in a subject, comprising: (a) a headset frame; (b) an electroencephalography (EEG) module, comprising two EEG electrodes; and (c) a transcranial direct current stimulation (tDCS) module, comprising three tDCS electrodes; (d) a wireless communication module, for communicating with at least one terminal and optionally at least one remote server; (f) a rechargeable battery and optionally a battery charging port, in electrical connection with EEG module and tDCS module; and (h) a processing unit configured to analyze the at least one set of EEG data and determine a tDCS protocol, wherein the EEG module is configured to obtain at least one set of EEG data of the subject; wherein the tDCS module is configured to stimulate at least one target brain region of the subject; wherein the wireless communication module is configured to transmit the at least one set of EEG data to a terminal or a remote server for analysis and configured to receive a tDCS protocol from the terminal or the remote server; and wherein the headset frame further comprises: (a1) a headband piece, comprising a central portion, a first arm portion, an opposing, second arm portion and a flexible, EEG supporting band extended from or attached to inner surface of the central portion, and the three EEG electrodes are positioned at the EEG supporting band, the headband piece that is sized and shaped to span at least around the T4 region and/or FP1 region the subject's head, wherein the central portion is configured to hold the three EEG electrodes; (a2) a tDCS electrode holding piece, comprising a lateral electrode holding portion and a neck portion, the tDCS electrode holding piece is configured to hold the two tDCS electrodes, wherein the neck portion is integral with or attached to the central portion at the lower side and is integral with or attached to the lateral electrode holding portion at the upper side; wherein the neck portion is configured to be switchable at least between a retracted position and an extended position; wherein, when the headset frame is worn on the head of the subject, the least one EEG electrodes are positioned proximate to or in contact with the FP1 region and/or FP2 region of the subject, and the at least one tDCS electrodes are positioned proximate to or in contact with F3 region and/or F4 region of the subject's head; wherein each of the tDCS electrodes comprises a plurality of conductive silicone electrodes arranged to form a comb-shaped or brush-shaped configuration each of the two tDCS electrodes comprises a plurality of conductive silicone electrodes arranged to form a comb-shaped or brush-shaped configuration, the two TDCS electrodes that are positioned at or proximate to each end of the lateral electrode holding portion and are slidably movable relative to the lateral electrode holding portion; and wherein the tDCS protocol is determined based on the analysis of the at least one set of EEG data, and the at least one tDCS module is configured to stimulate the at least one target brain region of the subject wearing the wearable device according to the tDCS protocol, and each comb-shaped or brush-shaped conductive silicone electrode configured to stimulate at least a portion of the target brain region of the subject with a current of magnitude around 1.5 mA to around 2 mA.

Embodiment 19. A system for assessing and/or modulating a neurological condition in a subject, comprising: (a) the at least one wearable device of embodiment 1; (b) at least one terminal; and (c) optionally, at least one remote server, wherein the at least one terminal and optionally the at least one remote server are in communication connection with each of the at least one wearable device, wherein the system is configured to: (i) analyze at least one set of EEG data obtained from the EEG module of the wearable device to generate at least one assessment of the neurological condition of the subject wearing the wearable device; and/or (ii) generate a tDCS protocol based on the at least one assessment, such that the tDCS protocol is customized according to the neurological condition of the subject, wherein the tDCS module is configured to simulate at least one target brain region of the subject according to the tDCS protocol.

Embodiment 20. The system of embodiment 19, wherein each of the remote server is a cloud-based platform in wireless communication with the at least one wearable device and/or the at least one terminal.

Embodiment 21. The system of any one of embodiment 19, wherein the neurological condition comprises cognitive impairment (COI), anxiety, depression and/or attention deficit hyperactivity disorder (ADHD).

Embodiment 22. A method for assessing and/or modulating a neurological condition in a subject, comprising the steps of: (a) obtaining, by at least one EEG module, at least one set of EEG data of the subject wearing the wearable device of any one of embodiments 1-18; (b) optionally, transmitting, by a wireless communication module of the wearable device, the at least one set of EEG data to a terminal or a remote server; (c) analyzing, by the at least one wearable device, the at least one terminal and/or the at least one remote server, the at least one set of EEG data to generate at least one assessment of the neurological condition of the subject; (d) generating, by the at least one wearable device, at least one terminal and/or the at least one remote server, a tDCS protocol based on the at least one assessment such that the tDCS protocol is customized according to the neurological condition of the subject; (e) optionally, transmitting, by the at least one terminal and/or the at least one remote server, the tDCS protocol to the wireless communication module of the wearable device; and (f) executing, by the tDCS module, the tDCS protocol to stimulate at least one target brain region of the subject.

Embodiment 23. The method of embodiment 22, further comprising the step of: (g) obtaining, by a heart rate sensor module, at least one heart rate data of the subject.

Embodiment 24. The method of embodiment 22, wherein step (f) further comprising the step of: (f1) stimulating, by the at least one tDCS module, the at least one target brain region of the subject with a current of magnitude around 1.5 mA to around 2 mA.

### EXAMPLES

The following example embodiments alone or in combination may be practiced to provide devices, systems, and methods for AI-based integrated diagnosis and therapeutic intervention of cognitive and neurophysiological conditions.

### Example 1

### 1. Devices

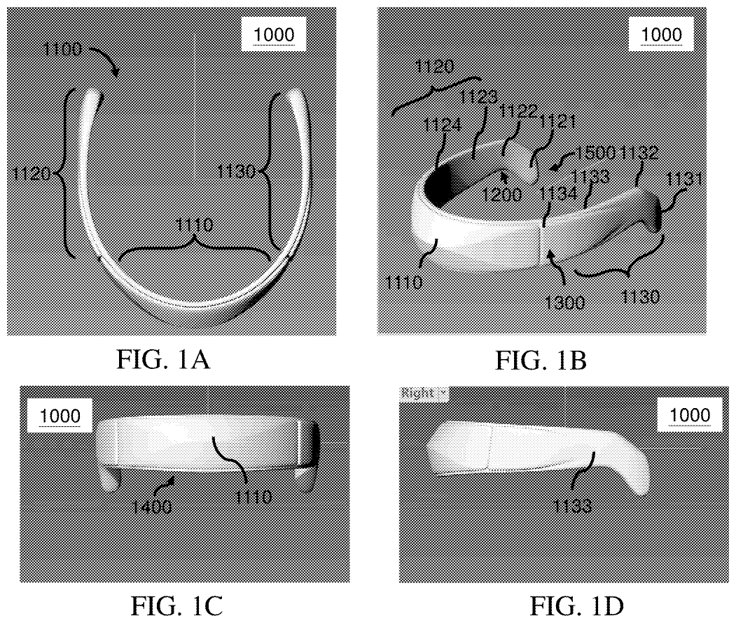

Referring now to FIGS. 1A-1D, showing an example embodiment of wearable device 1000 for assessing and modulating a neurological condition in a subject. In this example, the wearable device 1000 includes a headset frame 1100, electroencephalography (EEG) modules 1200, transcranial direct current stimulation (tDCS) modules 1300, and a wireless communication module 1400. The EEG modules 1200, the tDCS modules 1300, and the wireless communication module 1400 are disposed on or housed within the headset frame 1100 and are in communication connection with each other. The EEG modules 1200 are configured to obtain EEG data of the subject wearing the device (not shown in FIGS. 1A-1D). The tDCS modules 1300 are configured to stimulate at least one target brain region of the subject wearing the device 1000 according to a tDCS protocol, which is determined based on an analysis of the at least one set of EEG data.

Now referring to FIG. 1A, showing a top view of the wearable device 1000. In this embodiment, the headset frame 1100 is a headband-shaped housing or arcuate frame, shaped and sized to conform to the contour of the subject's head, extending from one side of the head to the other in a manner like a hairband. The headset frame 1100 is curved in shape, configured to rest over the parietal region or along the crown of the head, providing stable physical contact with the scalp when worn by the subject. The headset frame 1100 includes a center portion 1110 shaped and sized to rest on the top of the subject's head, a first arm portion 1120 extending posteriorly from a first side (hereinafter the right side) of the center portion 1110, and a second arm portion 1130 extending posteriorly from a second side (hereinafter the left side) opposite to the first side of the center portion 1110. The headset frame design allows for the secure attachment of the wearable device to fit the subject's head dimensions comfortably.

Now referring to FIG. 1B, showing a perspective view of the device 1000. The first arm portion 1120 includes a first arm distal portion 1121, a first arm bending portion 1122, a first arm elongated portion 1123, and a first arm proximal portion 1124. The first arm proximal portion 1124 connects the right side of the headset center portion 1110 with the first arm portion 1120. The first arm elongated portion 1123 extends posteriorly from the first arm proximal portion 1124 towards the first arm distal portion 1121, bending downwards at the first arm bending portion 1122 proximate to the first arm distal portion 1121. The first arm bending portion 1122 is shaped to conform to the shape of the subject's right ear for a comfortable fit. Symmetrically, the second arm portion 1130 includes a second arm distal portion 1131, a second arm bending portion 1132, a second arm elongated portion 1133, and a second arm proximal portion 1134 with similar shapes and structure as the first arm portion 1120. The second arm proximal portion 1134 connects the left side of the headset center portion 1110 with the second arm portion 1130. The second arm elongated portion 1133 extends posteriorly from the second arm proximal portion 1134 towards the second arm distal portion 1131, bending downwards at the second arm bending portion 1132 proximate to the second arm distal portion 1131. The second arm bending portion 1132 is shaped to conform to the shape of the subject's left ear for a comfortable fit.

In this embodiment, the tDCS modules 1300 are housed within the headset frame 1100 in a position proximate to the second arm proximal portion 1134, with the tDCS electrodes (not shown) disposed on the interior surface of the headset frame 1100 at a position proximate to the second arm proximal portion 1134 such that the tDCS electrodes can effective contact with the scalp of the subject wearing the wearable device 1000. In some embodiments, each tDCS electrode has a diameter of 5 mm or less. The EEG modules 1200 are housed within the headset frame 1100 in a position proximate to first arm bending portion 1122, with the EEG electrodes (not shown) disposed on the interior surface of the headset frame 1100 at a position proximate to first arm bending portion 1122 such that the EEG electrodes can effective contact with the scalp of the subject wearing the wearable device 1000. In some embodiments, each EEG electrode has a diameter of 5 mm or less.

In some embodiments, a battery charging port 1500 is disposed on the first arm distal portion 1121. In some embodiments, a power switch and/or an LED indicator displaying the battery status of the wearable device 1000 are disposed on the first arm portion 1120 or the second arm portion 1130.

In some embodiments, the plurality of tDCS electrodes are sized and shaped as an array of tDCS electrodes. In some embodiments, the plurality of EEG electrodes are sized and shaped as an assay of EEG electrodes.

Now referring to FIG. 1C, showing a front view of the device 1000. The wireless communication module 1400 is housed within the center portion 1110. In some embodiments, the center portion 1110 can also house one or more power cables and/or signal cables.

Now referring to FIGS. 1B-1D, multiple batteries are housed within the first arm elongated portion 1123, the second arm elongated portion 1133, and/or the center portion 1110. In some embodiments, the batteries are rechargeable batteries. In some embodiments, the batteries are generally rectangular in shape with a thickness of around 4 mm.

In some examples, the headset device is a wearable device. The EEG modules (also referred to as EEG sensors) are wireless hybrid EEG units which are configured to collect EEG signals at target brain regions. The tDCS electrodes are configured to deliver tDCS current to said regions.

In some examples, the device may further include a heart rate sensor module for measuring heart rate to obtain the heart rate data of the subject.

In some examples, the EEG sensors are configured to monitor absolute spectral power at certain frequency bands. The tDCS electrodes are configured to stimulate the target brain region at certain current (e.g. 1.5 mA-2 mA).

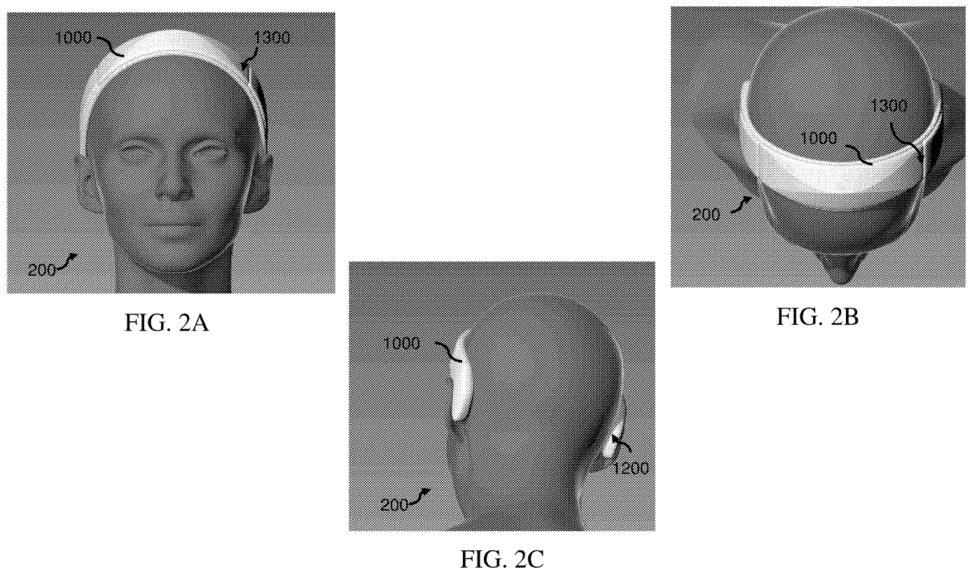

Now referring to FIGS. 2A-2C, showing the position of the wearable device 1000 when it is positioned on the head of the subject 200. The wearable device 1000 is configured to rest over the parietal region or along the crown of the head, providing stable contact with the scalp. This configuration provides stable and balanced placement of the wearable device 1000 on the subject's head, enabling EEG electrodes of the EEG modules 1200 and tDCS electrodes of the tDCS modules 1300 along the interior surface of the frame to effectively contact the targeted cranial regions.

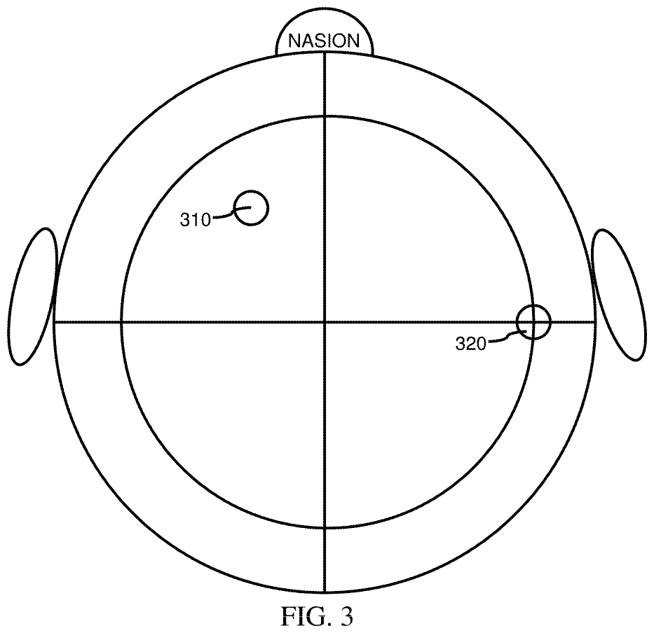

Now referring to FIGS. 2A-2C and FIG. 3 together. When the subject is wearing the wearable device 1000, the EEG electrodes of the EEG modules 1200 are positioned to effectively contact a specific region of the scalp that correspond to the T4 region 320 of the brain, such that the EEG modules 1200 can record EEG data from the T4 region 320 of the brain of the subject. The tDCS electrodes of the tDCS modules 1300 are positioned to effectively contact a specific region of the scalp that correspond to the F3 region 310 of the brain, such that the tDCS modules 1300 can stimulate the F3 region 310 of the brain of the subject.

In some examples, the EEG sensors and/or tDCS electrodes are positioned to monitor and/or stimulate certain brain regions, such as Dorsolateral Prefrontal Cortex (DLPFC).

In some embodiments, the device is configured to perform the following steps: (i) quantify cognition of the subject using electroencephalogram (EEG); (ii) identify EEG biomarkers indicative of cognitive impairment (COI); and (iii) apply a personalized Transcranial Direct Current Stimulation (tDCS) protocol targeting these EEG biomarkers.

### 2. Systems

In one example embodiment, the system is a closed-loop system including:
(i) a wearable device according to any of the embodiments as described herein that includes wireless hybrid EEG-tDCS electrodes for simultaneous EEG recording and tDCS delivery;
(ii) an AI-based data processing module that is connected to the headset device and is configured to detect EEG anomalies and adjusts treatment intensity in real time.

In some examples, the system includes a server configured to securely share data on EEG biomarkers (also referred to as “a set of EEG data” in some embodiments) and treatment with physicians (e.g. via one or more user terminals connected to the system), enabling remote monitoring. In some embodiments, by enhancing cognitive performance in depression subjects, the system is able to improve the adherence and efficacy of antidepressants, which are often hindered by poor working memory.

In some examples, the system includes a server configured to execute steps of a workflow according to an AI algorithm. The AI algorithm is configured to receive input data, analyze the data (e.g. for diagnosis), and output the intended data.

In some examples, the AI model and/or related algorithms may include any kind of existing AI models. In some examples, the AI models are trained by training data, which are labeled in a labelling process.

In some examples, the system generally includes a cloud server and/or clinician terminal for data storage, transmission and display. In some examples, the personalized/customized tDCS protocol is configured to be adjusted by using the data from the cloud server and/or clinician terminal (e.g. feedback by clinician).

In some embodiment, the system includes an infra-red lock housed within the wearable device that is configured to enhance the adherence of the subject as the clinician knows the identity of the subject using the wearable device.

In some examples, the system includes databases and software modules. The databases are configured to have data infrastructure for the collection of individual and population EEG biomarker data that enables personalized/customized NIBS treatment protocols. In some examples, the system includes subject-and physician-facing portals (e.g. mobile app) that are configured to enable remote monitoring.

In some examples, the system includes a server configured to execute program instructions of an AI algorithm that is trained using public and/or proprietary data to interpret EEG data collected and identify anomalies that contribute to subjects'COI symptoms. The AI algorithm forms a closed-loop system that adjusts treatment in real time based on EEG biomarkers.

### 3. Methods

In this example, provided is a method for assessing and modulating a neurological condition in a subject using the system of any one of the preceding claims, comprising the steps of:
(a) obtaining, by the at least one EEG module, at least one set of EEG data of the subject wearing the wearable device;
(b) transmitting, by the wireless communication module of the wearable device, the at least one set of EEG data to the server;
(c) analyzing, by the AI module, the at least one set of EEG data to generate at least one assessment of neurological condition of the subject;
(d) generating, by the AI module, the tDCS protocol based on the at least one assessment such that the tDCS protocol is customized according to the neurological condition of the subject;
(e) transmitting, by the server, the tDCS protocol to the wireless communication module of the wearable device; and
(f) executing, by the at least one tDCS module, the tDCS protocol to simulate the target brain region of the subject.

In some embodiments, the method further comprises the following steps prior to step (a):
obtaining at least one set of training EEG data from a reference data source labeled by a set of neurological conditions and/or a set of tDCS protocols;
training the AI module with the at least one set of training EEG data until a performance of the AI module meets a predetermined performance condition; and
deploying the AI module for performing steps (c) and/or (d).

In some embodiments, the method further comprises the step of obtaining, by a heart rate sensor module, at least one heart rate data of the subject.

In some embodiments, the method further comprises stimulating, by the at least one tDCS module, the target brain region of the subject with a current of magnitude around 1.5 mA to around 2 mA.

In some embodiments, the method further comprises the step of obtaining, by a heart rate sensor module, at least one heart rate data of the subject.

In some embodiments, the method further comprises the step of stimulating, by the at least one tDCS module, at least a portion of head area of the subject with a current of magnitude 1.5 mA-2 mA.

### Example 2.1

### 4. Example Wearable Device

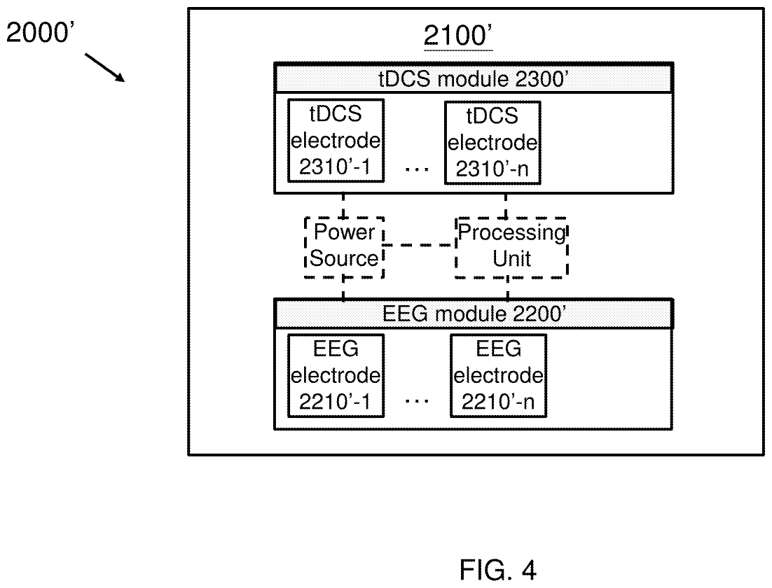

Now referring to FIG. 4, showing a schematic diagram of an example wearable device 2000′ for assessing and/or modulating a neurological condition in a subject. The device 2000′ generally includes a headset frame 2100′, an electroencephalography (EEG) module 2200′ and a transcranial direct current stimulation (tDCS) module 2300′. The EEG module 2200′ includes a plurality of EEG electrodes (or EEG sensors) 2310′ and the tDCS module includes a plurality of tDCS electrodes 2210′.

In some implementations, in addition to a plurality of EEG electrodes 2310′, the EEF module also generally includes signal amplifier configured to amplify and filter the received signals, a digital converter to digitize the conditioned signals and a microcontroller for data processing and output.

In some implementations, in addition to a plurality of tDCS electrodes 2210′, the DCS module also generally includes control and safety circuit (such as a microcontroller for ramping and monitoring impedance and automatic shutoff).

In one implementation, the tDCS module 2300′ includes two or more tDCS electrodes, including at least one anode and one cathode).

In one implementation, the device 2000′ further includes a power source (e.g., a battery) and/or a processing unit, which are in electrical connection with each other including the EEG module 2200′ and the tDCS module 2300′.

In another one implementation, the device 2000′ does not include the power source (e.g., a battery) or a processing unit. An external power source and/or external processing unit are in electrical connection with the device 2000′ instead.

### Example 2.2

### 5. Example Wearable Device

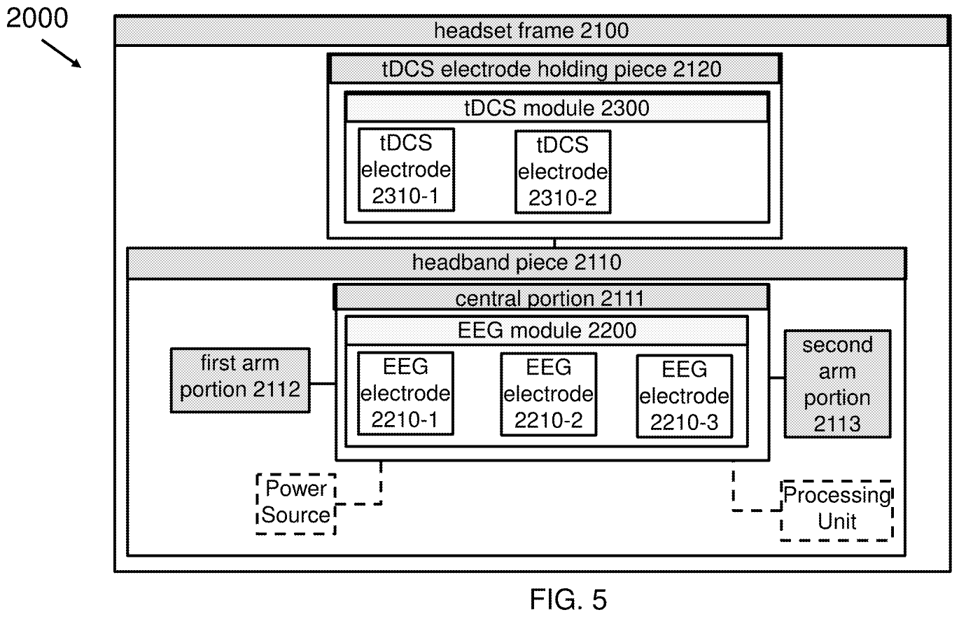

Now referring to FIG. 5, showing a schematic diagram of another example wearable device 2000 for assessing and/or modulating a neurological condition in a subject. The device 2000 generally includes a headset frame 2100, an electroencephalography (EEG) module 2200 and a transcranial direct current stimulation (tDCS) module 2300. The EEG module 2200 includes a plurality of EEG electrodes 2210 and the tDCS module includes a plurality of tDCS electrodes 2310. In this example, the EEG module 2200 includes three EEG electrodes 2210-1, 2210-2, 2210-3 (collectively as 2210) and the tDCS module includes two tDCS electrodes 2310-1 and 2310-2 (collectively as 2310). The EEG module is configured to obtain at least one set of EEG data of the subject through the plurality of EEG electrodes 2210. The tDCS module is configured to stimulate at least one target brain region of the subject. In this example, the at least one target brain region includes the second region of the subject's head.

In one implementation, the device 2000′ further includes a power source (e.g., a battery) and/or a processing unit, which are in electrical connection with each other as well as the EEG module 2200′ and the tDCS module 2300.

In another one implementation, the device 2000 does not include the power source (e.g., a battery) or a processing unit. An external power source and/or an external processing unit is connected with the device 2000.

The headset frame 2100 generally includes a headband piece 2110 and a tDCS electrode holding piece 2120. The headband piece 2110 and the tDCS electrode holding piece 2120 are either separate pieces that are directly or indirectly connected (or attached) with each other, or they are formed as one integral piece. In this example, the headband piece 2110 generally includes a central portion 2111, a first arm portion 2112 and an opposing, second arm portion 2113. The central portion 2111, the first arm portion 2112 and the second arm portion 2113 can be of any sizes and shapes, provided that together they form a headband that can be worn by the subject's head and that the central portion 2111 is proximate to the first region. In this example, the central portion 2111, the first arm portion 2112 and the second arm portion 2113 together form a generally U-shaped headband. The first arm portion 2112 and the second arm portion 2113 are either integral with, extended from or attached to the central portion 2111 at the two opposing ends of the central portion 2111. The headband piece 2110 that is sized and shaped to at least span around a first target region the subject's head, and it generally and partially conforms to contour of the subject's head. The central portion 2111 is configured to directly or indirectly hold the EEG electrodes 2310, which may be in electrical connection with a power source and a processing unit. The tDCS electrode holding piece 2120 is configured to directly or indirectly hold the tDCS electrodes. When the headset frame is worn on the subject's head, the EEG electrodes are positioned proximate to or in contact with the first target region of the subject's head, and the tDCS electrodes are positioned proximate to or in contact with the second target region of the subject's head.

In some implementations, the first region is or includes T4 region and/or FP1 region; and/or the second region is or includes F3 region and/or F4 region of the brain.

In one implementation, the headband piece 2110 is made of flexible material (such as soft plastic materials known in the art) such that it is flexibly conforms to contour of the subject's head. In another implementation, the headband piece 2110 is made of hard material (such as hard plastic).

### Example 3

### 6. Example Wearable Device Comprising Example EEG Module

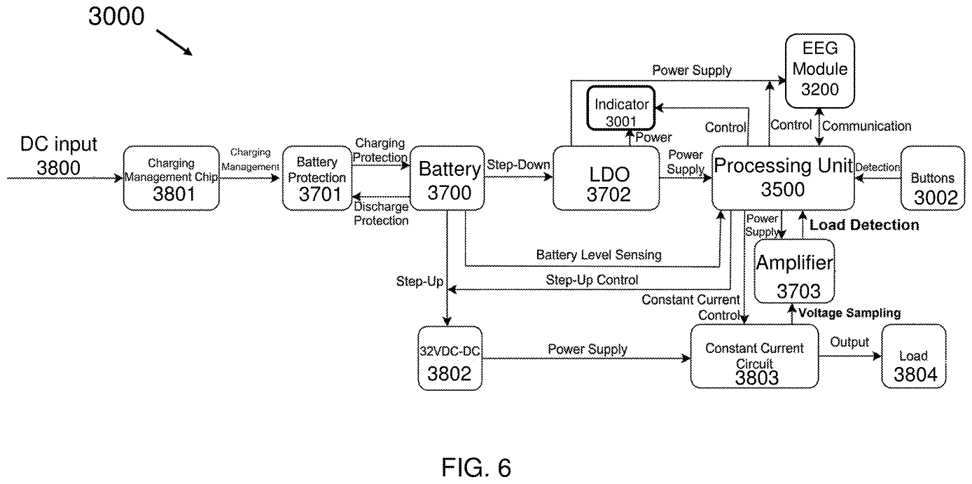

Now referring to FIG. 6, showing another example wearable device 3000 including an example EEG module 3200. In this example, in addition to the EEG module 3200, the example wearable device 3000 generally includes a processing unit 3500 and a battery 3700. The battery 3700 is in electrical connection with the other components to provide power supply. The EEG module 3200 is configured to operatively connect with and is in bidirectional data communication with the processing unit 3500, wherein the processing unit 3500 is configured to control over the EEG module 3200 by transmitting configuration parameters, and the EEG module 3200 is configured to obtain EEG data and send back to the processing unit 3500 for data processing, for assessing a neurological condition in a subject.

In one implementation, the wearable device 3000 further includes a Low Dropout Regulator (LDO) 3702, an indicator 3001, an amplifier 3703, one or more buttons 3002, a battery protection module 3701, a charging management chip 3801, a 32VDC-DC converter 3802, a constant current circuit 3803, and a load 3804. The LDO 3702 is in electrical connection with the battery 3700, the EEG module 3200, the indicator 3001 and the processing unit 3500. The LDO 3702 is a linear voltage regulator that can perform step-down voltage from the battery 3700 and provide a power supply from the battery 3700 while maintaining a stable output voltage 3700 to components such as the EEF module 3200 and the processing unit 3500, even when the input voltage is very close to the output voltage. The charging management chip 3801 is in electrical connection with the battery protection module 3701, which is in electrical connection with the battery 3700. In this example, the charging management chip 3801 is configured to receive DC input 3800 and is configured to manage the charging of the battery protection module 3701. The battery protection module 3701 is configured to protect charging and discharging of the battery 3700. The converter 3802 is configured to step up voltage from the battery and provide another power supply to the constant current circuit 3803, which also receives power supply from the processing unit 3500. The constant current circuit 3803 is configured to output to the load 3804 and provide voltage sampling to the amplifier 3703, which provides load detection to the processing unit 3500. The processing unit 3500 is configured to receive or detect input signal from buttons 3002 to trigger the operation, and also configured to control operation of the indicator 3001, to control step-up voltage of the converter 3802, and to control constant current to the constant current circuit 3803. The processing unit 3500 is also configured to receive or detect battery level and/or power output level of battery 3700. The processing unit 3500 is also configured to provide power supply to amplifier 3703, and to process load detection from amplifier 3703.

In some implementation, the device 3000 further includes a tDCS module that is configured to operatively connect with an independent processing unit and/or an independent battery.

### Example 4

### 7. Example Wearable Device Comprising Example EEG Module and TDCS Module

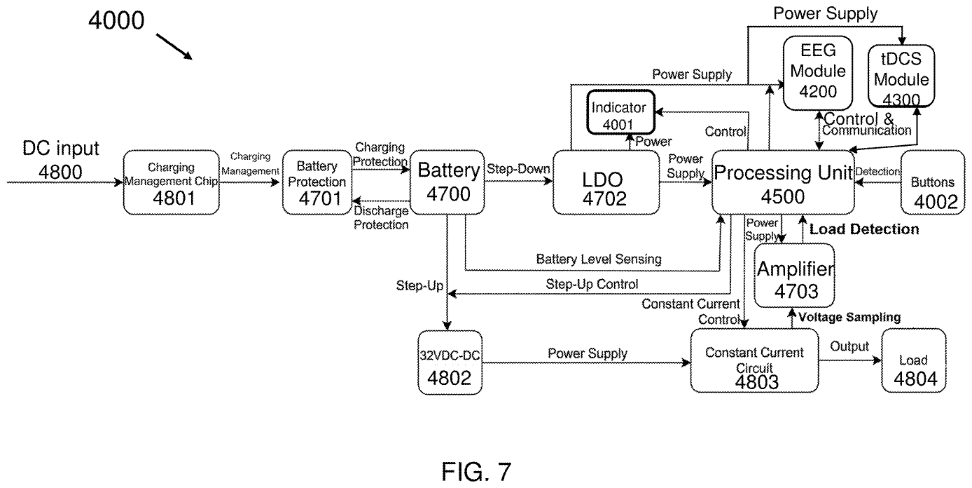

Now referring to FIG. 7, showing another example wearable device 4000 including an example EEG module 4200 and an example tDCS module 4300. Similar to Example 3, the example wearable device 4000 also includes a processing unit 4500 and a battery 4700. In one implementation, the wearable device 4000 further includes a Low Dropout Regulator (LDO) 4702, an indicator 4001, an amplifier 4703, one or more buttons 4002, a battery protection module 4701, a charging management chip 4801, a 32VDC-DC converter 4802, a constant current circuit 4803, and a load 4804. The detailed structure and configuration are similar to those described in Example 3 and thus not repeated herein for brevity's sake. In this example, the charging management chip 4801 is configured to receive DC input 4800 and is configured to manage the charging of the battery protection module 4701. In this example, the LDO 4702 also provides power supply from the battery 4700 to the tDCS module 4300. The tDCS module 4300 is also configured to operatively connect with and is in bidirectional data communication with the processing unit 4500, wherein the processing unit 4500 is configured to also control over the tDCS module 4300 by transmitting configuration parameters, and the tDCS module 4300 is configured to receive tDCS protocol from the processing unit 3500. The tDCS module 4300 is configured to provide electrical stimulation of at least one target brain region of the subject for modulating a neurological condition in the subject.

### Example 5

### 8. Example Wearable Device

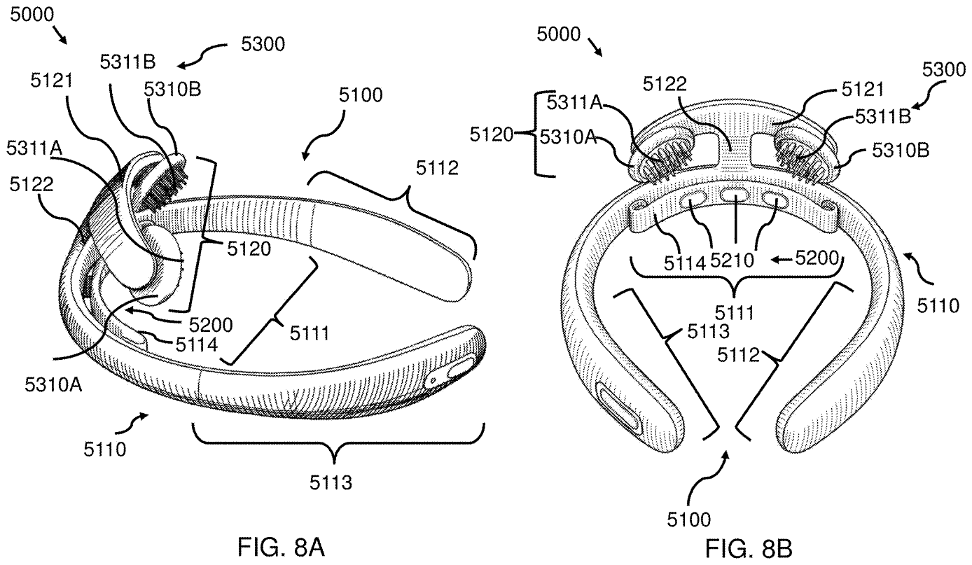

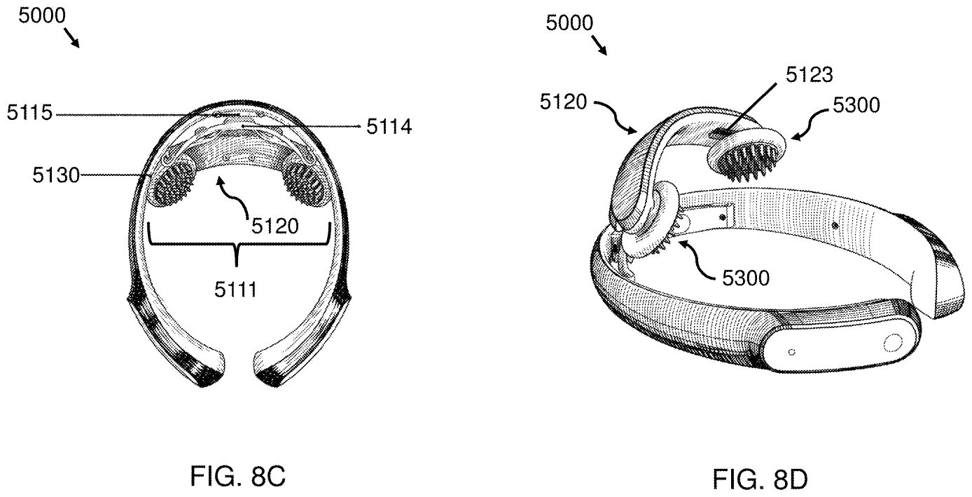

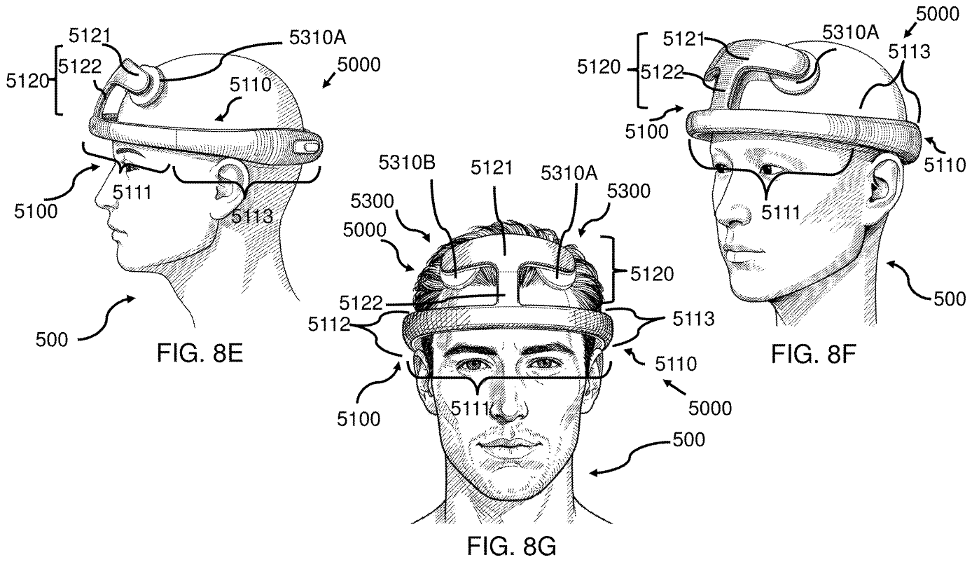

Referring now to FIGS. 8A-8D, showing another example embodiment of a wearable device 5000 for assessing and/or modulating a neurological condition in a subject. In this example, the wearable device 5000 generally includes a headset frame 5100, an electroencephalography (EEG) module 5200 that is configured to obtain at least one set of EEG data (from the first target region) of the subject and a transcranial direct current stimulation (tDCS) module 5300 is configured to stimulate at least one target brain region (i.e., the second target region) of the subject.

The headset frame 5100 is generally a headband-shaped housing or arcuate frame, shaped and sized to generally conform to the contour of the subject's head. The headset frame 5100 is sized and shaped such that is able to be worn on the head of a subject and includes a headband piece 5110 and a tDCS electrode holding piece 5120. In this example, the headset frame 5100 is curved in shape, configured to rest over the forehead region and along the crown of the head, providing stable physical contact with the forehead when worn by the subject. The headset frame design allows for the secure attachment of the wearable device to fit the subject's head dimensions comfortably.

The headband piece 5110 includes a central portion 5111, a first arm portion 5112 and a second arm portion 5113. In this example, the first arm portion 5112 and the second arm portion 5113 are extended from (and integral with) the central portion 5111 at the two opposing ends of the central potion 5111. These three portions are configured to form a U-shaped headband that is sized and shaped to at least span around a portion of the subject's head (and covers at least the first target region). The central portion 5111 is configured to hold the EEG module 5200 at the inner side, facing the subject's head. In this example, the central potion 5111 further includes a top supporting base 5115 and an EEG supporting band 5114 on the inner side. The EEG supporting band 5114 is made of flexible materials (such as silicone) and is configured to attach to the inner side of the central portion 5111. The EEG supporting band 5114 contains a middle portion attached to the top supporting base 5115, and two bent end portions attached to the two end portions of the middle portion (or proximate to the first arm portion or second arm portion). The top supporting base 5115 provides support to the flexible EEG supporting band 5114 and has a certain thickness to space the EEG supporting band 5114 away from the central portion 5111. The EEG electrodes 5210 are provided on the inner side of the EEG supporting band, and are operatively connected with the central portion 5111 through the top supporting base 5115. As such, the EEG supporting band provides flexible contact of the EEG electrodes with the subject's head, accommodating various sizes and shapes of subject's heads. In this example, the top supporting base 5115 is made of hard plastics such as Acrylonitrile Butadiene Styrene (ABS). In this example the EEG supporting band 5114 is made of soft materials such as silicone.

The tDCS electrode holding piece 5120 is configured to hold the tDCS electrodes 5310A and 5310B (collectively 5310) of the tDCS module 5300. The tDCS electrode holding piece 5120 is generally a T-shaped piece that includes a lateral electrode holding portion 5121 and a neck portion 5122. The neck portion 5122 is longitudinally extended from or is attached to the top side of the central portion 5111, and is bent toward the inner side (i.e., toward subject's head). The neck portion 5122 is configured to be switchable between at least a retracted position and an extended position so as to allow longitudinal adjustment of the position of the tDCS electrodes proximate to the target region (second target region) of the subject's head for adopting different sizes and shapes. In one implementation, the means for switching between the retracted position and the extended position includes a telescopic sliding mechanism, wherein the neck portion includes an inner member slidably received within an outer member, and a friction fit or spring-loaded detent releasably locks the neck portion at a selected position. Other mechanisms known in the part may be used instead.

The lateral holding portion 5121 is extended from or attached to the neck portion 5122 and is bent to conform to the subject's head. In this example, the tDCS electrodes 5311A and 5311B (collectively 5311) contain a plurality of conductive silicone electrodes arranged to form a comb-shaped or brush-shaped configuration and are received in disc-shaped tDCS electrode sockets 5310A and 5310B (collectively 5310), respectively. In one implementation, the at least one tDCS electrodes are configured to be slidably movable relative to the lateral electrode holding portion. Each of the tDCS electrode sockets 5310 is movably connected to the lateral holding portion 5121 by sliding rail 5123, so as to allow lateral adjustment of the position of the tDCS electrodes proximate to the target region (second target region) of the subject's head for accommodating different sizes and shapes.

Now referring to FIG. 8E, FIG. 8F and FIG. 8G, showing a subject 500 wearing the wearable device 5000. In this example, the EEG electrodes are adjusted and positioned proximate to or in contact with the first target region (i.e., forehead, including FP1 and/or FP2 regions) to collect at least one set of EEG data. The tDCS electrodes are adjusted and positioned proximate to or in contact with the second target region (i.e., F3 and/or F4 regions) to stimulate at least one target brain region of the subject.

In some further implementations, the headset frame of the wearable device 5000 further includes a bent portion (e.g., similar to example 1) and contains EEG electrodes at the second arm portion so as to also target the T4 region of the subject to collect at least one set of EEG data. In other implementations, the headset frame has other sizes and shapes to adopt for targeting other desired target regions of the target's head.

In some implementations, the wearable device also include a central control unit (a microprocessor), a wireless communication module (such as a Wi-Fi module and/or a Bluetooth module), a battery, a status sensor (such as an infrared sensor), etc. In this example, the battery is a 300 mAh battery that is chargable through a Type-C port or magnetic port. In one implementation, the wearable device 5000 also includes an indicator, such as a RGB light. In one implementation, the tDCS electrodes of the tDCS module 5311 provides a current of magnitude around 1.5 mA to around 2 mA to the subject. In this example, the current of magnitude of tDCS module 5311A is adjustable. In this example, the central control unit is a microcontroller unit (MCU) which stores and processes data from the wearable device 5000, transmits and receives data wirelessly, and controls the overall operation of the device, including operation of the EEG module and current of magnitude of tDCS module.

In some implementations, the wearable device has the following technical specifications:

### 1. Product Overview

Product use: Suitable for head massage therapy to relieve fatigue.
Intended users: General users.
Operator: User.
Places of use: Home and outdoor use.
Use environment: Normal environment.
Number of modes: 1.
Number of channels: Single channel.
Display screen: None.
Indicator light: RGB.
Product color: Dark blue and light blue.
Battery capacity: 300 mAh.
Charging interface: Type-C.

### 2. Description of Product Parts

### Parts

Button: Power button.
Display/indicator area: Indicator light.
Output end: Comb-shaped electrode.

### 3. Power Supply Method

The product uses DC 5V charging.
The charging interface is described here as magnetic charging.
Recommended charger input: 5V, 500 mA or above.
Charging protection voltage range: 4.8V to 5.2V.

### 4. Size and Weight

Product size: 201.8×163.2 ×92.6 mm.
Product weight: About 180 g.

### 5. Product Functions

### Button Function

The main unit has one button.
Press and hold for 1 second to turn it on.
Press and hold for 1 second to turn it off.

### Indicator Function

The product uses an RGB indicator light.
Low battery: Yellow light stays on for 10 seconds, then the device shuts down.
While charging: Red light stays on.
When fully charged: Green light stays on.
During operation: Green light flashes.

### Timer Function

The default operating time is 15 minutes, and timing starts only when output is active.
After the timer ends, the device turns off automatically.
If there is no output, the device will automatically turn off after 5 minutes of idle time.
For a low-voltage alarm, the warning lasts 10 seconds, and then the device turns off automatically.
The output ramp-up time can be adjusted through the app.
Adjustable range: 0 to 600 seconds.
Default ramp-up time: 30 seconds.
It cannot be adjusted while output is running.

### Battery Level Detection and Low Battery Warning

If battery voltage is 3.9V or above, battery level is more than 75%.
If battery voltage is below 3.9V but at least 3.6V, battery level is 50% to 75%.
If battery voltage is below 3.6V but at least 3.3V, battery level is 25% to 50%.
If battery voltage is below 3.3V, battery level is below 25%, and a low-voltage warning is triggered.

### Bluetooth Function

The main unit has Bluetooth capability.
The mobile app will be developed by the customer.
A Bluetooth communication protocol is provided.
An OTA (over-the-air) upgrade function is reserved, so the device firmware can be updated through Bluetooth.

### 6. Product Parameter Requirements

### Battery

Battery specification: 300 mAh.
Charging current: 258 mA±10%.
Charging time: About 2 hours from low battery to full charge.

### Static Current Requirement

When the product is turned off, the battery discharge current (static current) must be less than 30 uA.

### Output Parameters

1 channel, 1 mode, 4 intensity levels.
Default setting after power-on is level 0.
Pulse frequency: None.
Pulse width: None.
Output waveform: None.
Load detection: Not available at present.
The hardware has reserved this function, but the software has not yet been designed for it.
Output intensity requirement: with a load of 10 kΩ or less, the constant current maximum is 2 mA, with an allowed error of 10%.

### 7. Operation and Instructions for Use

The button is used only for turning the device on and off.
All other control functions are handled through the app.
The main unit supports 4 intensity levels: 0.5 mA, 1 mA, 1.5 mA, and 2 mA.
The app supports start, pause, and ramp-up time adjustment.
An OTA function is reserved so the internal firmware can be upgraded remotely through the app.
After output starts, the intensity gradually rises to the target output current.
Ramp-up time can be adjusted from 0 to 600 seconds.
Default ramp-up time is 30 seconds.

### 8. Environmental Conditions

### Operating Conditions

Ambient temperature: +5 °C to +40 °C
Relative humidity: 30% RH to 75% RH.
Atmospheric pressure: 700 hPa to 1060 hPa.

### Transportation and Storage Conditions

Ambient temperature: −20 °C to +55 °C
Relative humidity: 10% RH to 90% RH.
Atmospheric pressure: 500 hPa to 1060 hPa.

### Example 6

### 9. Example Systems

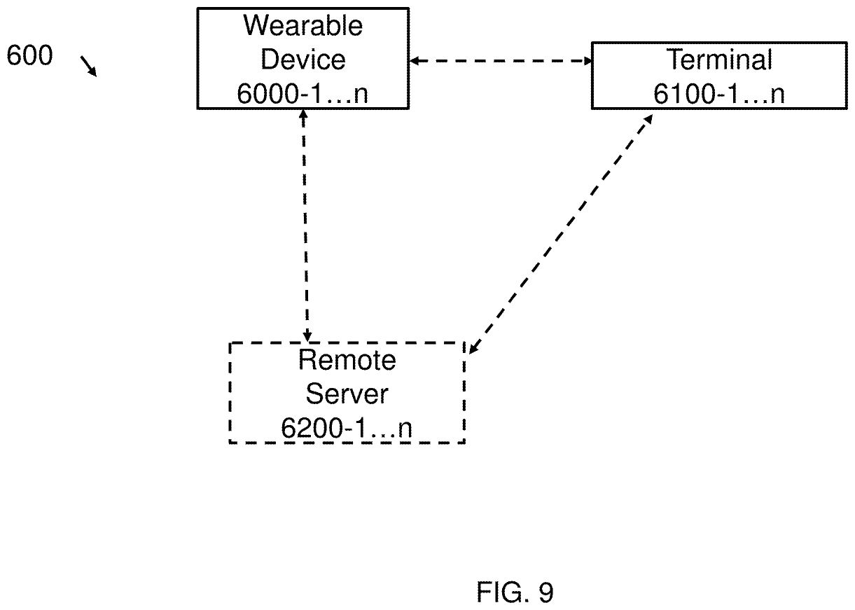

Now referring to FIG. 9A, showing an example system 600 for assessing and/or modulating a neurological condition in a subject. The system 600 generally contains at least one or more example wearable devices 600-1 . . . n (collectively as 6000) as described in any one of the previous examples, and at least one or more terminals 6100-1 . . . n (collectively as 6100). In some implementations, each of the wearable devices 6000 contains an EEG module, a tDCS module and a wireless communication module which is configured to wirelessly communicate with each of the terminals 6100.

In one implementation, the wearable device 6000 also contains a microprocessor, the least one set of EEG data obtained from the EEG module of the wearable device 6000 is analyzed in the microprocessor to generate at least one assessment of the neurological condition of the subject wearing the wearable device. The microprocessor is configured to generate a tDCS protocol based on the at least one assessment, such that the tDCS protocol is customized according to the neurological condition of the subject. The tDCS module is configured to simulate at least one target brain region (second region) of the subject according to the tDCS protocol. In this implementation, the terminal 6100 is served as a user interface (e.g., through an APP) to control the wearable device 6000, to receive, transmit and/or display the results including the at least one set of EEG data, at least one assessment and the tDCS protocol. In some other implementations, the at least one set of EEG data is transmitted to the terminal 6100 for analysis, wherein the at least one assessment is partially or entirely generated by the terminal, and the tDCS protocol is partially or entirely generated by the terminal 6100. The tDCS protocol is then transmitted to the wearable device 6000 for execution by the tDCS module to stimulate the target brain region of the subject. In one further implementation, the terminal 6100 is a smartphone, tablet, a personal computer and/or a dedicated remote control.

In some implementations, the system 600 further contains one or more remote server 6200. In one further implementation, the one or more remote server 6200 is in wireless communication with each of the wearable device 6000 and/or the at least one terminal 6100. The at least one set of EEG data is directly or indirectly transmitted to the remote server 6200 for analysis, wherein the at least one assessment is partially or entirely generated by the remote server 6200, and the tDCS protocol is partially or entirely generated by the remote server 6200. The tDCS protocol is then directly or indirectly transmitted to the wearable device 6000 for execution by the tDCS module to stimulate the target brain region of the subject. In some further implementations, the remote server further comprises an AI module as described in any one of the previous examples.

Example 7

### 10. Example Methods

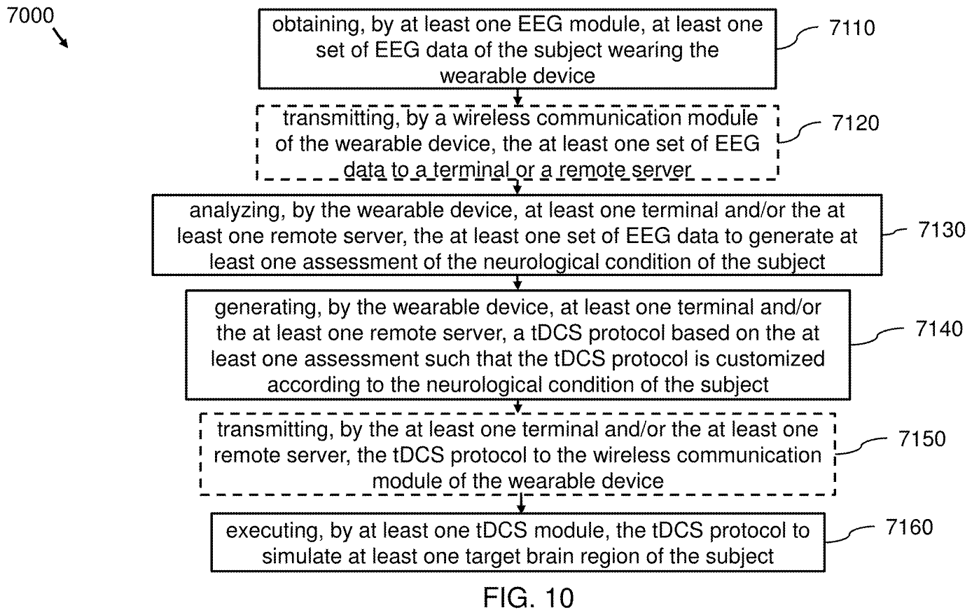

Now referring to FIG. 10, showing an example method 7000 for assessing and/or modulating a neurological condition in a subject using a wearable device or system as described in any one of the previous examples. The method 700 includes the steps of:
(a) obtaining, by at least one EEG module, at least one set of EEG data of the subject wearing the wearable device of any one of claims 1-18;
(b) optionally, transmitting, by a wireless communication module of the wearable device, the at least one set of EEG data to a terminal or a remote server, if present;
(c) analyzing, by the at least one wearable device, the at least one terminal and/or the at least one remote server, the at least one set of EEG data to generate at least one assessment of the neurological condition of the subject;
(d) generating, by the at least one wearable device, at least one terminal and/or the at least one remote server, a tDCS protocol based on the at least one assessment such that the tDCS protocol is customized according to the neurological condition of the subject;
(e) optionally, transmitting, by the at least one terminal and/or the at least one remote server, if present, the tDCS protocol to the wireless communication module of the wearable device; and
(f)executing, by the tDCS module, the tDCS protocol to stimulate at least one target brain region of the subject.

In one implementation, the method 7000 further includes the step of:
(g) obtaining, by a heart rate sensor module, at least one heart rate data of the subject.

In one implementation, the step (f) further includes the step of:
(f1) stimulating, by the at least one tDCS module, the at least one target brain region of the subject with a current of magnitude around 1.5 mA to around 2 mA.

The system and method of the present disclosure may be implemented in the form of a software application running on a computer system. Further, portions of the methods may be executed on one such computer system, while the other portions are executed on one or more other such computer systems. Examples of the computer system include a mainframe, personal computer, handheld computer, server, etc. The software application may be stored on a recording media locally accessible by the computer system and accessible via a hard wired or wireless connection to a network, for example, a local area network, or the Internet.

The computer system may include, for example, a processor, random access memory (RAM), a printer interface, a display unit, a local area network (LAN) data transmission controller, a LAN interface, a network controller, an internal bus, and one or more input devices, for example, a keyboard, mouse etc. The computer system can be connected to a data storage device.

Blocks and/or methods discussed herein can be executed and/or made by a user, a user agent (including machine learning agents and intelligent user agents), a software application, an electronic device, a computer, firmware, hardware, a process, a computer system, and/or an intelligent personal assistant. Furthermore, blocks and/or methods discussed herein can be executed automatically with or without instruction from a user.

It should be understood for those skilled in the art that the division between hardware and software is a conceptual division for ease of understanding and is somewhat arbitrary. Moreover, it will be appreciated that peripheral devices in one computer installation may be integrated to the host computer in another. Furthermore, the application software systems may be executed in a distributed computing environment. The software program and its related databases can be stored in a separate file server or database server and is transferred to the local host for execution.

The exemplary embodiments of the present invention are thus fully described. Although the description referred to particular embodiments, it will be clear to one skilled in the art that the present invention may be practiced with variation of these specific details. Hence this invention should not be construed as limited to the embodiments set forth herein.

Methods discussed within different figures can be added to or exchanged with methods in other figures. Further, specific numerical data values (such as specific quantities, numbers, categories, etc.) or other specific information should be interpreted as illustrative for discussing example embodiments. Such specific information is not provided to limit example embodiment.

What is claimed is:

1. A wearable device for assessing and/or modulating a neurological condition in a subject, comprising:
(a) a headset frame;
(b) an electroencephalography (EEG) module, comprising at least one EEG electrodes; and
(c) a transcranial direct current stimulation (tDCS) module, comprising at least one tDCS electrodes,
wherein the EEG module is configured to obtain at least one set of EEG data of the subject;
wherein the tDCS module is configured to stimulate at least one target brain region of the subject; and
wherein the headset frame further comprises:
(a1) a headband piece, comprising a central portion, a first arm portion and a second arm portion, the headband piece that is sized and shaped to at least span around a first region the subject's head, wherein the central portion is configured to hold the at least one EEG electrodes;
(a2) a tDCS electrode holding piece, configured to hold the at least one tDCS electrodes, and
wherein, when the headset frame is worn on the head of the subject, the least one EEG electrodes are positioned proximate to or in contact with the first target region, and the at least one tDCS electrodes are positioned proximate to or in contact with a second target region of the subject's head.

2. The wearable device of claim 1, wherein the first region is or comprises T4 region, FP1 region and/or FP2 region; and/or the second region is or comprises F3 region and/or F4 region.

3. The wearable device of claim 1, wherein the tDCS electrode holding piece comprises a lateral electrode holding portion and a neck portion;
wherein the neck portion is integral with or attached to the central portion at the lower side and is integral with or attached to the lateral electrode holding portion at the upper side; and
wherein the neck portion is configured to be switchable at least between a retracted position and an extended position.

4. The wearable device of claim 1, further comprising a wireless communication module for communicating with at least one terminal and/or at least one remote server.

5. The wearable device of claim 4, wherein the wireless communication module is configured to transmit the at least one set of EEG data to the at least one terminal and/or the at least one remote server for analysis and is configured to receive a tDCS protocol from the at least one terminal and/or the at least one remote server; and
wherein the tDCS protocol is determined based on the analysis of the at least one set of EEG data.

6. The wearable device of claim 5, wherein the at least one tDCS module is configured to stimulate the at least one target brain region of the subject wearing the wearable device according to the tDCS protocol.

7. The wearable device of claim 1, further comprising a flexible, EEG supporting band extended from or attached to inner surface of the central portion, and wherein the at least one EEG electrodes comprise three EEG electrodes that are positioned at the EEG supporting band.

8. The wearable device of claim 3, wherein the at least one tDCS electrodes comprise two TDCS electrodes that are positioned at or proximate to each end of the lateral electrode holding portion.

9. The wearable device of claim 1, wherein each of the tDCS electrodes comprises a plurality of conductive silicone electrodes arranged to form a comb-shaped or brush-shaped configuration.

10. The wearable device of claim 3, wherein the at least one tDCS electrodes are configured to be slidably movable relative to the lateral electrode holding portion.

11. The wearable device of claim 1, wherein the at least one tDCS module comprises a plurality of tDCS electrodes, each configured to stimulate at least a portion of the target brain region of the subject with a current of magnitude around 1.5 mA to around 2 mA.

12. The wearable device of claim 1, wherein the neurological condition comprises cognitive impairment (COI), anxiety, depression and/or attention deficit hyperactivity disorder (ADHD).

13. The wearable device of claim 5, further comprising a processing unit configured to analyze the at least one set of EEG data and determine the tDCS protocol.

14. The wearable device of claim 1, further comprising a heart rate sensor module configured to obtain at least one set of heart rate data of the subject.

15. The wearable device of claim 1, further comprising a rechargeable battery and optionally a battery charging port in electrical connection with the EEG module and the tDCS module.

16. The wearable device of claim 1, further comprising a wear status module configured to determine a wear status of the wearable device, wherein the wear status comprising an on-head status and an off-head status of the subject.

17. The wearable device of claim 16, wherein the wear status module comprising an infra-red sensor.

18. A wearable device for assessing and/or modulating a neurological condition in a subject, comprising:
(a) a headset frame;
(b) an electroencephalography (EEG) module, comprising two EEG electrodes; and
(c) a transcranial direct current stimulation (tDCS) module, comprising three tDCS electrodes;
(d) a wireless communication module, for communicating with at least one terminal and optionally at least one remote server;
(f) a rechargeable battery and optionally a battery charging port, in electrical connection with EEG module and tDCS module; and
(h) a processing unit configured to analyze the at least one set of EEG data and determine a tDCS protocol,
wherein the EEG module is configured to obtain at least one set of EEG data of the subject;
wherein the tDCS module is configured to stimulate at least one target brain region of the subject;
wherein the wireless communication module is configured to transmit the at least one set of EEG data to a terminal or a remote server for analysis and configured to receive a tDCS protocol from the terminal or the remote server; and
wherein the headset frame further comprises:
(a1) a headband piece, comprising a central portion, a first arm portion, an opposing, second arm portion and a flexible, EEG supporting band extended from or attached to inner surface of the central portion, and the three EEG electrodes are positioned at the EEG supporting band, the headband piece that is sized and shaped to span at least around the T4 region and/or FP1 region the subject's head, wherein the central portion is configured to hold the three EEG electrodes;
(a2) a tDCS electrode holding piece, comprising a lateral electrode holding portion and a neck portion, the tDCS electrode holding piece is configured to hold the two tDCS electrodes,
wherein the neck portion is integral with or attached to the central portion at the lower side and is integral with or attached to the lateral electrode holding portion at the upper side;
wherein the neck portion is configured to be switchable at least between a retracted position and an extended position;
wherein, when the headset frame is worn on the head of the subject, the least one EEG electrodes are positioned proximate to or in contact with the FP1 region and/or FP2 region of the subject, and the at least one tDCS electrodes are positioned proximate to or in contact with F3 region and/or F4 region of the subject's head;
wherein each of the tDCS electrodes comprises a plurality of conductive silicone electrodes arranged to form a comb-shaped or brush-shaped configuration each of the two tDCS electrodes comprises a plurality of conductive silicone electrodes arranged to form a comb-shaped or brush-shaped configuration, the two TDCS electrodes that are positioned at or proximate to each end of the lateral electrode holding portion and are slidably movable relative to the lateral electrode holding portion; and
wherein the tDCS protocol is determined based on the analysis of the at least one set of EEG data, and the at least one tDCS module is configured to stimulate the at least one target brain region of the subject wearing the wearable device according to the tDCS protocol, and each comb-shaped or brush-shaped conductive silicone electrode configured to stimulate at least a portion of the target brain region of the subject with a current of magnitude around 1.5 mA to around 2 mA.

19. A system for assessing and/or modulating a neurological condition in a subject, comprising:
(a) the at least one wearable device of claim 1;
(b) at least one terminal; and
(c) optionally, at least one remote server,
wherein the at least one terminal and optionally the at least one remote server are in communication connection with each of the at least one wearable device,
wherein the system is configured to:
(i) analyze at least one set of EEG data obtained from the EEG module of the wearable device to generate at least one assessment of the neurological condition of the subject wearing the wearable device; and/or
(ii) generate a tDCS protocol based on the at least one assessment, such that the tDCS protocol is customized according to the neurological condition of the subject,
wherein the tDCS module is configured to simulate at least one target brain region of the subject according to the tDCS protocol.

20. The system of claim 19, wherein each of the remote server is a cloud-based platform in wireless communication with the at least one wearable device and/or the at least one terminal.

21. The system of claim 19, wherein the neurological condition comprises cognitive impairment (COI), anxiety, depression and/or attention deficit hyperactivity disorder (ADHD).

22. A method for assessing and/or modulating a neurological condition in a subject, comprising the steps of:
(a) obtaining, by at least one EEG module, at least one set of EEG data of the subject wearing the wearable device of claim 1;
(b) optionally, transmitting, by a wireless communication module of the wearable device, the at least one set of EEG data to a terminal or a remote server;
(c) analyzing, by the at least one wearable device, the at least one terminal and/or the at least one remote server, the at least one set of EEG data to generate at least one assessment of the neurological condition of the subject;
(d) generating, by the at least one wearable device, at least one terminal and/or the at least one remote server, a tDCS protocol based on the at least one assessment such that the tDCS protocol is customized according to the neurological condition of the subject;
(e) optionally, transmitting, by the at least one terminal and/or the at least one remote server, the tDCS protocol to the wireless communication module of the wearable device; and
(f) executing, by the tDCS module, the tDCS protocol to stimulate at least one target brain region of the subject.

23. The method of claim 22, further comprising the step of:
(g) obtaining, by a heart rate sensor module, at least one heart rate data of the subject.

24. The method of claim 22, wherein step (f) further comprising the step of:
(f1) stimulating, by the at least one tDCS module, the at least one target brain region of the subject with a current of magnitude around 1.5 mA to around 2 mA.
